(19) United States
(12) Patent Application Publication
(10) Pub. No.: US 2025/0051687 A1
(43) Pub. Date: Feb. 13, 2025

(54) MALONATE AND FURAN BASED SURFACTANTS

(71) Applicant: Nederlandse Organisatie voor toegepast-natuurwetenschappelijk onderzoek TNO, 's-Gravenhage (NL)

(72) Inventor: Marc CROCKATT, 's-Hertogenbosch (NL)

(21) Appl. No.: 18/711,534

(22) PCT Filed: Nov. 18, 2022
(86) PCT No.: PCT/NL2022/050666

§ 371 Date: May 17, 2024

(30) Foreign Application Priority Data
Nov. 18, 2021 (EP) 21209082.3

(51) Int. Cl. C11D 1/66

(52) U.S. Cl. CPC C11D 1/66

(57) ABSTRACT

The invention is directed to a method for the preparation of a surfactant, said method comprising reacting a furanic compound according to formula (V), or a salt thereof, with a malonate according to formula (VI), or a salt thereof, to obtain a first furan-based surfactant precursor of formula (I) or a salt thereof,

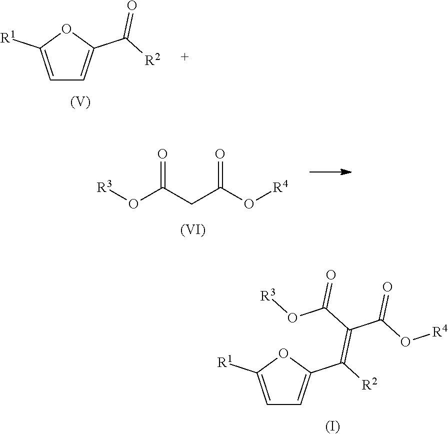

wherein R¹ is H or CH₂OX, wherein X is H or an aliphatic group;
wherein R², R³ and R⁴ are independently H or an aliphatic group;
wherein the method further comprises one or more steps to convert the first furan-based surfactant precursor of formula (I), or a salt thereof, to a surfactant, which surfactant comprises
a furan-moiety comprising a backbone structure of formula (VII),

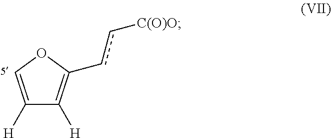

a hydrophilic moiety, preferably selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium and non-ionic groups, and;
a hydrophobic moiety comprising an aliphatic chain comprising at least 6 carbon atoms.

The invention is in the field of surfactants. In particular, the invention is directed to a method to prepare furan-based surfactants from furanic compounds and malonate-based compounds.

Surfactants, or surface-active agents, are chemical compounds that are capable of lowering the surface tension between two liquids or between a gas and a liquid. Surfactants are widely used for a range of applications, such as detergents, emulsifiers, foaming agents and dispersants. Typically, the compounds are amphiphilics. i.e. compounds with both a hydrophobic tail and a hydrophilic head. This combination may allow for a water-soluble component and a water-insoluble (i.e. oil-soluble) component. Accordingly, the surfactant may absorb at i.a. oil/water interphases and can provide interesting properties for a variety of applications.

It is expected that the market for surfactants undergoes continuous growth in the coming years. The main contributor to the market constitutes detergents and cleaners for households as over 50% of all surfactants typically end up in these products. Currently, the largest production comprises petrochemical-based linear alkylbenzene sulfonates (LAS), with around 4 million tons per year being produced and consumed.

LAS surfactants are typically a mixture of compounds based on a hydrophilic sulfonate head attached to a hydrophobic benzene ring which is attached to a hydrophobic alkyl chain. The big advantage of LAS is its tolerance of hard water, which commonly inhibits the functioning of other groups of surfactants. Further. LAS typically has a low critical micelle concentration, low Krafft temperature, fast wetting and good foaming behavior. LAS is accordingly suitable for broad use in a variety of applications.

However. LAS is fossil-based and has a negative environmental impact. Accordingly, there is a strong desire to provide more sustainable surfactants which have comparable or improved properties compared to LAS.

One method to provide surfactants produced from bio-based chemicals such as sugar-derived furans is described in e.g. WO2017/079718 and by Park et al. (ACS Cent. Sci. 2016, 2, 11, 820-824). Here, oleo-furan surfactants derived from furan and fatty acids are described. By adjusting the fatty acid the surfactant properties could be altered. Additionally, the surfactants could better tolerate the metal ions contained in hard water, therefore making chelating agents, which are typically added to e.g. detergents, unnecessary. However, the surfactants are synthesized from furan. While it is a bio-based molecule, it requires decarbonylation of furfural to prepare furan, thereby decreasing the atom efficiency and resulting in a waste stream of carbon monoxide. Furthermore, it requires an additional processing step, resulting in increased cost and inefficiency. Moreover, the process may be challenging to scale-up.

Other bio-based surfactants are described by Kipshagen et al. (Green Chem. 2019, 21, 3882). Here i.a. furfural and 5-hydroxymethylfurfural (5-HMF) are used as the basis for the surfactants. In the case of furfural, it is converted into tetrahydrofuran followed by side-chain manipulation and the introduction of a hydrophilic head. In the case of HMF, it is first hydrogenated to 2,5-bis(hydroxymethyl) furan (BHMF) followed by side-chain manipulation and introduction of the hydrophilic head.

5-HMF has also been used as a starting material for surfactants as described in WO2016/028845, wherein the final surfactants were prepared by esterification, amination and alkylation to produce non-ionic surfactants. However, these do not mimic LAS.

Other surfactants are described in e.g. WO2021/083642, WO2015/084813 and WO2017/079719.

A reaction of 5-(1-octynyl) furan-2-carboxaldehyde with diethylmalonate to prepare pharmaceutical compositions containing aromatic heterocyclic carboxylic acid derivatives is described in EP0194093.

It is an object of the present inventors to provide an improved method to prepare furan-based surfactants that overcomes at least part of the above-mentioned drawbacks. The present inventors realized that this can be achieved by a process comprising a reaction of a furanic compound and malonic acid or an ester thereof. It was found that this approach advantageously provides access to surfactants with a variety of hydrophobic tails and hydrophilic heads, enabling fine-tuning of the furan-based surfactant specific to the particular application of the surfactant. The method according to the present invention is further advantageously efficient and suitable to be scaled-up.

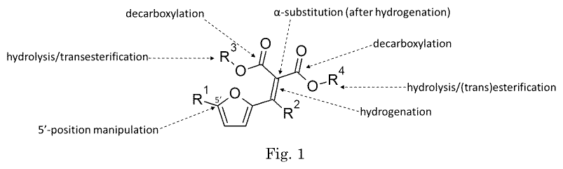

FIG. 1 illustrates a number of reactive sites of a furan-based surfactant precursor according to the present invention.

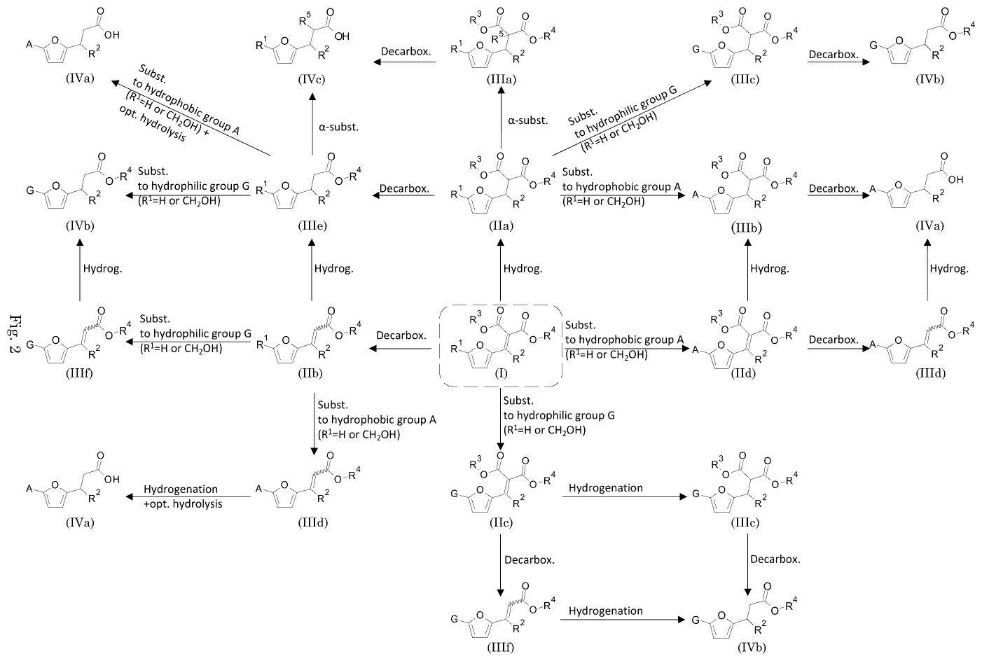

FIG. 2 illustrates a schematic overview of the possible reaction paths according to preferred embodiments of the present invention.

Accordingly, in a first aspect the present invention is directed to a method for the preparation of a surfactant, said method comprising reacting a furanic compound according to formula (V) with a malonate according to formula (VI) to obtain a first furan-based surfactant precursor of formula (I),

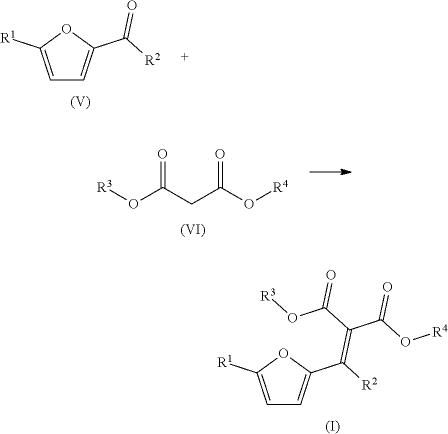

wherein R¹ is H or CH₂OX, wherein X is H or an aliphatic group;
wherein R², R³ and R⁴ are independently H or an aliphatic group.
   The method further comprises one or more steps to convert the first furan-based surfactant precursor of formula (I) to a surfactant, which comprises a furan-moiety, a hydrophilic group and a hydrophobic moiety.

The furan moiety of the surfactant comprises a backbone structure of formula (VII).

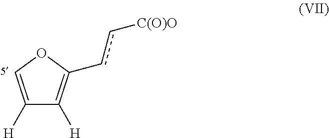

With “backbone structure” is meant that the formula schematically represents a core structure which may be optionally substituted with any group or atom at any position that does not specifically contain all substituents. Hence, more precisely, the backbone structure of formula (VII) may be substituted at any one or more of the positions indicated with an asterisk (*) in formula (VII) as below.

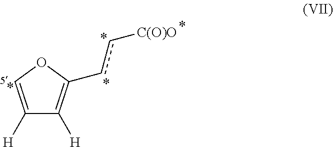

The dashed bond in formula (VII) means that this bond is optionally present and that the carbon atoms between the furanic ring and the C(O)O can be bound to each other by a single or double bond. This double bond may be cis or trans orientated as the backbone structure represents any and all isomers, including regio-isomers, diastereomers and the like.

In other words, the surfactant comprises a furan-moiety of formula VIIa of which the 2′-position is substituted with a group comprising a —C—C—C(O)O— moiety of which the first C is bound to the furan ring, and of which the 3′-position and the 4′-position are H and the 5′-position is optionally substituted.

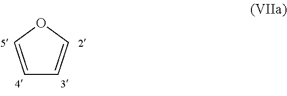

The reaction of the furanic compound and the malonate according to the present invention can also generally be referred to as a condensation reaction or a Knoevenagel-type condensation. Suitable methods and reaction conditions for the Knoevenagel-type condensation are disclosed in WO 2018/236218. Van Schijndel et al. Synlett 29 (2018) 15, 1983-1988, and Song et al. Catalysts 6 (2016) 106.

Aliphatic group is herein used to describe aliphatic groups comprising one or more carbon atoms. e.g. methyl, ethyl, propyl and the like. The aliphatic group may be an aliphatic chain. Aliphatic chain is herein used to describe a carbon chain comprising multiple carbon atoms and may be linear, branched, cyclic, saturated and/or unsaturated but not aromatic. Thus, the carbons in the aliphatic chain may be joined by single bonds, one or more carbon pairs may be joined by a double bond and/or one or more carbons may be joined by a triple bond. Also, the chain may comprise one or more non-aromatic carbocyclic moieties. In case the aliphatic chain is branched or cyclic, the main chain (i.e. the longest chain of carbon atoms) is considered the backbone chain. The hydrophilic tail may suitably be an aliphatic hydrocarbon chain or an aliphatic ether. Preferably, if the aliphatic chain comprises a certain number of carbon atoms. e.g. at least 6 carbon atoms, it is preferred that the backbone chain comprises said number of carbon atoms. e.g. at least 6 carbon atoms. The branch may on itself also be an aliphatic chain.

The terms polar, apolar, hydrophilic and hydrophobic are commonly used in the field of surfactants. It is commonly known that a surfactant is amphiphilic and comprises hydrophobic (or apolar) and hydrophilic (or polar) moieties. In general, hydrophilic is used to describe the capacity of a molecular entity or of a substituent to interact with polar solvents, in particular with water, or with other polar groups and the tendency to mix, be wetted and/or dissolve in water. Typically, hydrophobic is used for moieties that form Van der Waals bonds and minimal to no hydrogen bonds. For instance, aliphatic chains may be considered hydrophobic, while sulfonic and carboxylic acid groups are considered hydrophilic.

The hydrophilic group that is part of the surfactant in accordance with the present invention is preferably selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium and non-ionic groups.

The furan compound according to formula (V) is preferably furfural (i.e. R¹ and R² are H). HMF (i.e. R¹ is CH₂OH and R² is H), or a derivative of these.

In particular embodiments. R² of the furan compound according to formula (V) is an aliphatic group. Such a furan compound can suitably be prepared from a precursor according to formula (V) wherein R² is H, followed by an oxidation, as illustrated in Scheme 1, wherein R¹ is H or CH₂OX, wherein X is H or an aliphatic group and R² is an aliphatic group.

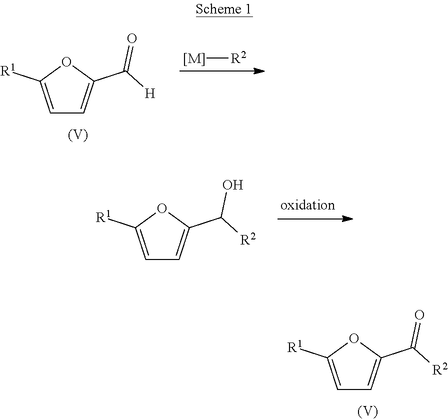

The addition of the aliphatic group R² as illustrated in Scheme 1 can be carried out using well-known chemistry, including for example reaction with an organometallic nucleophile ([M]-R²) in the Grignard reaction or the related Barbier reaction (see e.g. Clayden et al. Organic Chemistry, Oxford University Press and WO 2015/084813). The oxidation to obtain (V) in Scheme 1 can for instance be carried out using well-known chemistry (see i.a. Clayden et al. Organic Chemistry. Oxford University Press), but also using more specialized catalyzed processes as described in Ye et al., Chem. Commun . . . 56 (2020) 11811-11814.

In particular embodiments, the furan compound of formula (V) has an R¹ that is CH₂OX wherein X is an aliphatic group. Such a compound can be obtained as a product from a dehydration and hydrolysis process of a sugar, wherein an alcohol is applied in situ. Alternatively, such a compound can be obtained by alkylating a precursor according to formula (V) wherein R¹ that is CH₂OH, as illustrated in Scheme 2, with a compound X-Y wherein X is an aliphatic group and Y is a leaving group. e.g. a halide such a bromide. Alkylation of alcohols is also well-known chemistry (see i.a. Clayden et al. Organic Chemistry, Oxford University Press).

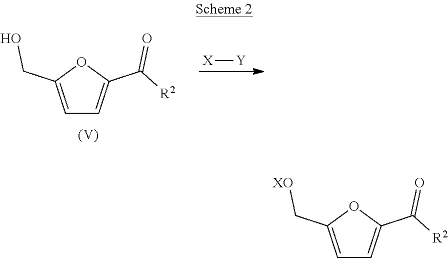

The malonate compound according to formula (VI) can be malonic acid (i.e. R⁸ and R⁴ are H), a monoester (i.e. R⁸ or R⁴ is H and R⁸ or R⁴ is an aliphatic group) or a diester (i.e. R⁸ and R⁴ are both an aliphatic group, which may be the same of different). Malonic acid and various mono- and diester are commercially available and others can easily be prepared using standard esterification methods known to the skilled person.

The present inventors found that the first furan-based surfactant precursor of formula (I) is multifunctional, meaning it can be manipulated in a variety of reaction processes to readily access a large variety of surfactant products. As illustrated in FIG. 1, these reaction processes include hydrogenation, decarboxylation, 5′-position manipulation, hydrolysis, esterification and/or transesterification, and hydrogenation followed by α-substitution. A combination of these processes, either in one or in subsequent reaction steps is also possible. The 5′-position for the 5′-position manipulation may be found by counting the number of atoms in the furan ring, starting at the oxygen as 1′-position, the carbon atom substituted with the —C—C—C(O)O— moiety as the 2′-position and so forth, up to the fifth atom in the furan ring, i.e. the 5′-position. Further, an α-carbon can be identified as the carbon adjacent to the one or more carbonyl groups. The α-substitution accordingly occurs at this α-carbon. These positions are present in the structure of formula (I) as illustrated below.

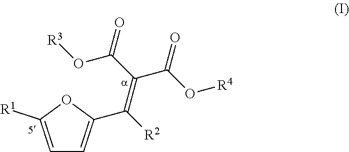

The present inventors recognized that the one or more steps to convert the first furan-based surfactant precursor of formula (I) may be one, two or three steps selected from the group consisting of hydrogenation, decarboxylation, 5′-position substitution or manipulation, and α-substitution. More steps are theoretically possible, but realistically not feasible in view of overall process efficiency and/or economics. However, hydrolysis, esterification and/or transesterification of the —CO₂R⁸ and —CO₂R⁴ are relatively facile steps and can be carried out in addition to said group of reactions.

Accordingly, as illustrated in FIG. 2, the first furan-based surfactant precursor of formula (I) may be converted in one, two or three step into the compounds included in Table 1. These compounds are referred herein to first, second and third-level compounds, depending on the number steps is required to prepare the compounds from the first furan-based surfactant precursor (excluding hydrolysis, esterification and/or transesterification).

TABLE 1
second, third and fourth level compounds
Second-level compounds:

| | |
|---|---|
| 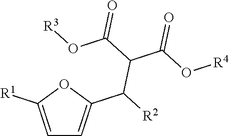 | (IIa) |
| 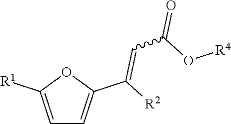 | (IIb) |
| 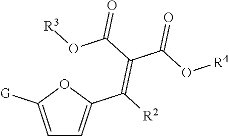 | (IIc) |
| 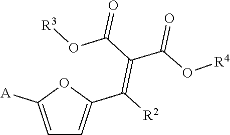 | (IId) |

Third-level compounds:

| | |
|---|---|
| 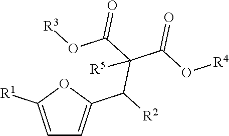 | (IIIa) |
| 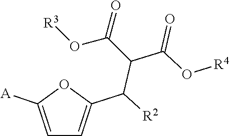 | (IIIb) |
| 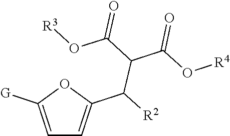 | (IIIc) |
| 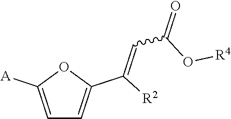 | (IIId) |
| 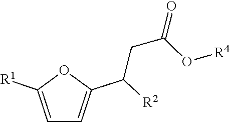 | (IIIe) |
| 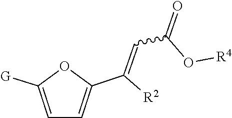 | (IIIf) |

Fourth-level compounds:

| | |
|---|---|
| 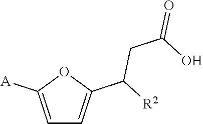 | (IVa) |
| 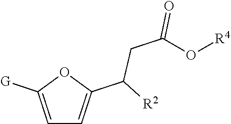 | (IVb) |
| 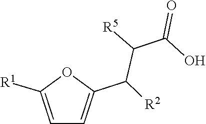 | (IVc) |

The labels A and G in any of the formulae in Table 1 represent hydrophobic and hydrophilic substituents, respectively. This is irrespective of whether the compound concerned can act as the surfactant or requires one or more further steps before the compound can act as such. This may be differed however for the substituents which are represented by R¹-R⁵. These substituents can suitably be selected based on whether the compound concerned can act as the surfactant, or whether the compound is continued in one or more further steps before the surfactant is formed. Namely, if the compound can act as the surfactant, it comprises both a hydrophilic and a hydrophobic moiety. On the other hand, if the compound is continued in one or more further steps, the substituents which are represented by R¹-R⁵ can suitably be converted or introduced (e.g. by hydrolysis, esterification, transesterification and the like) to obtain a compound that can act as a surfactant in a later stage. In the embodiments wherein the compound of any of the formulae in Table 1 is the surfactant according to the present invention, the options that R¹-R⁵ can be are thus restricted vis-à-vis the options for formula (I). On the other hand, in the embodiments wherein the compound is continued in one or more steps to obtain the surfactant, R¹-R⁵ can be as follows for any of formulae (IIa)-(IId) and (IIIa)-(IIIf): R¹ is H or CH₂OX, wherein X is H or an aliphatic group; R², R⁸ and R⁴ are independently H or an aliphatic group; and R⁵ is an aliphatic group.

Thus, in particular embodiments, the surfactant in accordance with the present invention has any of the formulae included in Table 2.

TABLE 2
Surfactants

| Formula | Substituents |
|---|---|
| 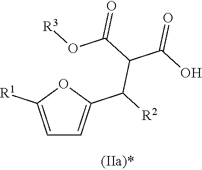 | R¹ is H or CH₂OX, wherein X is H or an aliphatic group, R² and R³ are independently H or an aliphatic group, provided that that at least one of R1-R3 is an aliphatic chain comprising more than 6 carbon atoms. |
| 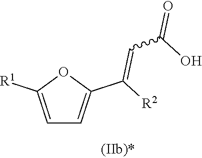 | R¹ is H or CH₂OX, wherein X is H or an aliphatic group, R² is H or an aliphatic group, provided that at least one of R¹ and R² is an aliphatic chain comprising more than 6 carbon atoms. |
| 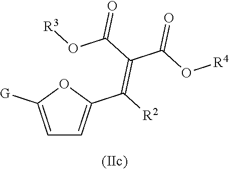 | R², R³ and R⁴ are indepen- dently H or an aliphatic group, provided that at least one of R², R³ and R⁴ is an aliphatic chain comprising more than 6 carbon atoms. |
| 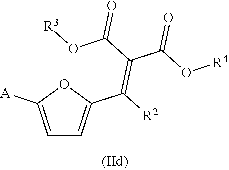 | R², R³ and R⁴ are indepen- dently H or an aliphatic group,provided that at least one of R³ and R⁴ is H. |
| 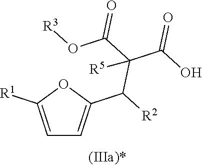 | R¹ is H or CH₂OX, wherein X is H or an aliphatic group, R² and R³ are independently H or an aliphatic group, R⁵ is an aliphatic chain com- prising more than 6 carbon atoms. |
| 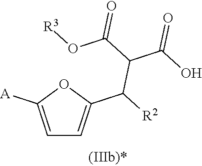 | R² and R³ are independently H or an aliphatic group. |
| 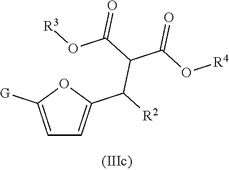 | R², R³ and R⁴ are indepen- dently H or an aliphatic group, provided that at least one of R², R³ and R⁴ is an aliphatic chain comprising six or more carbon atoms. |
| 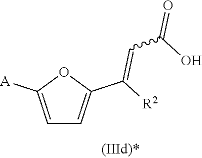 | R² is H or an aliphatic group. |
| 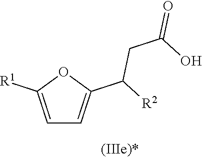 | R¹ is H or CH₂OX, wherein X is H or an aliphatic group, R² is H or an aliphatic group, provided that at least one of R¹ and R² is an aliphatic chain com- prising six or more carbon atoms. |
| 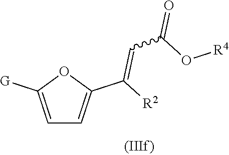 | R² and R⁴ are independently H or an aliphatic group, provided that at least one of R² and R⁴ is an aliphatic chain comprising six or more carbon atoms. |
| 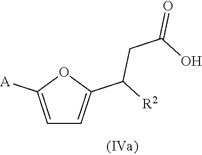 | R² is H or an aliphatic group. |
| 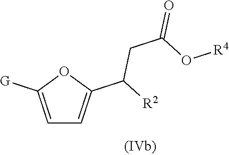 | R² and R⁴ are independently H or an aliphatic group, provided that at least one of R² and R⁴ is an aliphatic chain comprising six or more carbon atoms. |
| 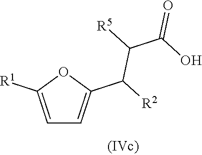 | R¹ is H or CH₂OX, wherein X is H or an aliphatic group, R² is H or an aliphatic group, R⁵ is an aliphatic chain comprising more than 6 carbon atoms. |

*R⁴ is H.

It may be appreciated that in the embodiments wherein the compound of any of formulae (IIa)-(IId) and (IIIa)-(IIIf) is the surfactant, said compound comprises a hydrophilic moiety represented by G and/or by at least one carboxylate originating from the malonate (i.e. R⁴ and/or R³ are/is H), the latter being particularly preferred. The hydrophilic moiety preferably originates from the malonate for sake of atom efficiency. More preferably, the hydrophilic group is a dicarboxylate originating from the malonate (i.e. R⁴ and R³ are H), for an even better atom efficiency. Accordingly, the surfactant is preferably of any of formulae (IIa), (IIIa) and (IIIb), with R³ and R⁴ both being H. The formulae (IIa) and (IIIb), with R³ and R⁴ both being H are most preferred in this respect. Accordingly, a method including providing (I), and converting (I) into (IIa) or (IIIb), optionally via (IId) is a particularly preferred embodiment.

For any of formulae (IIa)-(IId), (IIIa)-(IIIf) and (IVa)-(IVc), G represents a hydrophilic moiety. In particular embodiments wherein R¹=H (e.g. if furfural is used as a starting material), G may represent the hydrophilic group. In particular embodiments wherein R¹=CH₂OH, (e.g. if HMF is used as a starting material), G may represent a —CH₂-hydrophilic group.

The hydrophilic moiety may thus be an ionic moiety. e.g. an anionic, a cationic, or a non-ionic moiety. Examples of suitable non-ionic moieties that G may comprise include poly(ethylene oxide), poly(co-ethylene oxide co-propylene oxide) (also referred to as poloxamers), polyglycosides, isosorbide and its derivatives, 1,4-sorbitane and its derivatives, and the like. These moieties can readily be introduced by a reaction of the hydroxyl group of the first furan-based surfactant precursor (also referred to as the first level compound), and of the second and third level compounds wherein R¹ is CH₂OH.

The hydrophilic group is preferably an ionic group and can be present with a counter-ion to balance the charges. It may be appreciated that the counter-ions can be any ion that balances the charge, for instance, if the hydrophilic group has a monovalent negative charge, the counter-ion can be e.g. sodium (Na⁺), potassium (K⁺), lithium (Lit) and/or ammonium (NH₄⁺). Preferably, the counter-ion is an ammonium ion, an alkali metal ion or alkaline earth metal ion. Suitable hydrophilic groups and counter-ions are for instance described in WO2017/079719. The hydrophilic group, and preferably G as such, is preferably selected from the group consisting of sulfate (—O—SO₃⁻), sulfonate (—SO₃⁻), sulfinate (—SO₂⁻), thiosulfate (—O—S₂O₂⁻), sulfamidate (—NH—SO₃⁻), carboxylate (−CO₂⁻), sarcosinate and taurate (—NR—R—CO₂⁻), phosphate (—O—PO₃⁻ or —O—PO₂—OR⁻), pyrophosphate (—O—PO₂—O—PO₂—OR₂⁻), phosphonate (—PO₂R⁻ or —PO₃⁻), amines or ammonium (—NR₃⁺), polyammonium (—NR₂—R—NR₃²⁺)·hydroxyammonium (—NR₂OH⁺)·pyridinium (—NC₅H₅⁺), picolinium (—NC₅H₅—R⁺), imidazolinium (

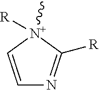

), benzimidazolinium (

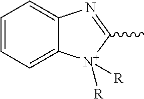

), oxonium (—OR₂⁺), sulfonium (—SR₂⁺) and phosphonium (—PR₃⁺). Herein [symbol] represents the bond to the furanic ring and R is H or a C₁-C₃ alkyl. Particularly good surfactant properties are obtained for sulfonate as hydrophilic group.

In a particular embodiment, the first level compound is such that R¹ is CH₂OH, which can directly or indirectly be manipulated to a hydrophilic group G. In such embodiments. G may be selected from the group consisting of methylene sulfate (—CH₂O—SO₃⁻), methylene sulfonate (—CH₂SO₃⁻), methylene sulfinate (—CH₂SO₂⁻), methylene thiosulfate (—CH₂O—S₂O₂⁻), methylene sulfamidate (—CH₂NH—SO₃⁻), methylene carboxylate (—CH₂CO₂⁻), methylene sarcosinate and taurate (—CH₂NR—R—CO₂⁻)·methylene phosphate (—CH₂O—PO₃⁻— or —CH₂O—PO₂—OR⁻), methylene pyrophosphate (—CH₂O—PO₂—O—PO₂—OR²⁻), methylene phosphonate (—CH₂PO₂R⁻ or —CH₂PO₃⁻)·methylene amines or ammonium (−CH₂NR₃⁺), methylene polyammonium (—CH₂NR₂—R—NR₃²⁺), methylene hydroxyammonium (—CH₂NR₂OH⁺), methylene pyridinium (—CH₂NC₅H₅⁺), methylene picolinium (—CH₂NC₅H₅-R³⁰), methylene imidazolinium (

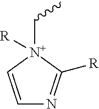

), methylene benzimidazolinium (

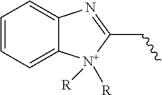

), methylene oxonium (—CH₂OR₂⁺), methylene sulfonium (—CH₂SR₂⁺) and methylene phosphonium (—CH₂PR₃⁺), wherein R is H or a C₁-C₃ alkyl, preferably H or Me.

For any of formulae (IIa)-(IId), (IIIa)-(IIIf) and (IVa)-(IVc). A represents a hydrophobic moiety. The hydrophobic moiety comprises an aliphatic chain. Notably. R² and R⁶ may also comprise an aliphatic chain, even if the compound comprises substituent A, in which embodiments the compound thus comprises more than one aliphatic chain. Unless explicitly specified differently herein, an aliphatic chain preferably comprising at least 4 carbon atoms, more preferably at least 6 carbon atoms, even more preferably between 6 to 26 carbon atoms, most preferably between 6 and 18 carbon atoms. Aliphatic chains comprising at least 6 carbon atoms can suitably be used as hydrophobic tails.

The first level compound of formula (I), and in particular embodiments also the second and/or third level compound of any of formulae (IIa)-(IId) and (IIIa)-(IIIf) is continued on one or more steps to obtain the surfactant. These steps preferably comprise hydrogenation, decarboxylation. 5′-position manipulation, preferably with a reactant comprising a hydrophobic group, hydrogenation followed by α-substitution or a combination thereof. And in addition to these reactions, hydrolysis, esterification and or transesterification may be carried out to manipulate the R³ and R⁴ groups. These steps and conversions are generally well-established and basic principle well-known, see e.g. Clayden et al. Organic Chemistry. Oxford University Press.

The hydrogenation can be carried out with the compound of any of formulae (I), (IIa)-(IId) and (IIIa)-(IIIf) that comprises a C—C double bond, i.e. (I). (IIb), (IIc), (IId), (IIId) and (IIIf), leading to a second, third or fourth level compound of formula (IIa), (IIIe), (IIIc), (IIId), (IVa) or (IVb), respectively. Suitable methods and reaction conditions are for instance those disclosed in Coutant et al Beilstein J. Org. Chem. 14 (2018) 2853-2860.

The hydrogenation can be optionally followed by an α-substitution, which can be carried out with the compound of any of formulae (I). (IIa)-(IId) and (IIIa)-(IIIf) of which the C—C double bond is reduced, but it preferably carried out with the compound of any of formulae (IIa) and (IIIe), leading to a third or fourth level compound of formula (IIIa) or (IVc), respectively. Suitable methods and reaction conditions for the α-substitution are for instance those disclosed in Arai et al. Tetrahedron Letters 51 (2010) 1273-1275 and Schelkun et al. Bioorg. Med. Chem. Lett. 16 (2006) 2329-2332.

The decarboxylation can be carried out with the compound of any of formulae (I), (IIa)-(IId) and (IIIa)-(IIIf) that comprises a β-diester moiety. i.e. (I). (IIa), (IIc), (IId), (IIIa), (IIIb) and (IIIc), leading to a second, third or fourth level compound of formula (IIb), (IIIe), (IIIf), (IIId), (IVc), (IVa) and (IVb), respectively. Suitable methods and reaction conditions are for instance those disclosed in Mohite and Bhat Org. Lett. 15 (2013) 17, 4564-4567 and Schuppan Chem. Commun. (2004) 792-793.

The 5′-position manipulation can be carried out with the compound of any of formulae (I), (IIa)-(IId) and (IIIa)-(IIIf) that comprises a nucleophilic site on the 5′-position of the furan. i.e. (I), (IIa), (IIb), (IIIa) and (IIIe), provided that R¹ of these compounds is H or CH₂OH. For embodiments wherein said compound has an R¹ that is H, the furan ring is relatively activated and nucleophilic, which allows a nucleophilic substitution of the 5′-position of the furan ring. For embodiments wherein said compound has an R¹ that is CH₂OH, the alcohol group is relatively activated and nucleophilic, which allows a manipulation of the 5′-position of the furan ring. In both of these types of embodiments, introduction of a hydrophobic (A) or a hydrophilic moiety (G) is possible. Suitable methods and reaction conditions for the instruction of a hydrophobic moiety such as an aliphatic group is are for instance those disclosed in Asta et al. Green Chem. 13 (2011) 3066-3069.

The skilled person can suitably select appropriate reactions to introduce the hydrophilic group to the appropriate first, second or third level compound of any of formulae (I). (IIa). (IIb). (IIIa) and (IIIe), provided that R¹ of these compounds is H or CH₂OH. For example, carboxylate or sulfonate as the hydrophilic group may be introduced by reacting the appropriate first, second or third level compound with carbon dioxide or sulfur trioxide, respectively (see also Sung Park et al. ACS Cent. Sci. 2 (2016) 11, 820-824. For the embodiments wherein R¹ is CH₂OH, the hydroxyl can act as a nucleophile to suitably introduce the hydrophilic group.

The hydrolysis, esterification and/or transesterification can be carried out with the compound of any of formulae (I). (IIa)-(IId) and (IIIa)-(IIIf) in any stage of the method. Accordingly, it may be appreciated if the first level compound is converted into a second level compound, which is then optionally further converted into a third level compound, which is in turn then also optionally further converted into a fourth level compound, the R⁸ and/or R⁴ group of the subsequent first, second, third and fourth level compound do not necessarily have to be the same. For example, in a particular embodiment, the first level compound may be according to formula (I) wherein R⁸ and R⁴ are both H, and R⁸ and R⁴ are converted in a aliphatic group (i.e. esterification) in a further stage of the overall method to prepare the surfactant. And vice versa, in another particular embodiment, the first level compound may be according to formula (I) wherein R⁸ and R⁴ are both Me, and R⁸ and R⁴ are converted in H (i.e. hydrolysis) in a further stage of the overall method to prepare the surfactant. In yet another exemplary embodiment, the first level compound may be according to formula (I) wherein R³ and R⁴ are both Me, and R⁶ and R⁴ are converted in an aliphatic chain (i.e. transesterification) in a further stage of the overall method to prepare the surfactant. Esterification, transesterification and hydrolysis can be carried out in, situ with the other one or more step described herein to prepare the surfactant. For example, the decarboxylation of β-diester moiety to obtain a mono-ester a may comprise hydrolysis as well. Suitable conditions for the hydrolysis and (trans) esterification are disclosed in WO 2018/236218.

FIG. 2 illustrates an overview of the possible reaction pathways that can be taken to convert the first furan-based surfactant precursor of formula (I). It may however be appreciated that any of the routes and formulae are individually according to the present invention. Also, a plurality of routes to a particular compound is according to the present invention. For the purpose of clarity and a concise description, features, routes, compounds and formulae are described herein as part of the same or separate embodiments, however, it will be appreciated that the scope of the invention may include embodiments having combinations of all or some of the features described.

It may be appreciated that any of the compounds, including the first, second, third and fourth level compounds, as well as the surfactant described herein may be in a salt form. In particular the compounds comprising an ionic group (e.g. carboxylate, sulfonate, etc.) may be in a salt form with a counter ion. Further, it may be appreciated that any of the compounds may be a single stereoisomer, a mixture of stereoisomers, a single regio-isomer or a mixture of regio-isomers, whenever applicable. Thus, if not explicitly indicated differently, each formula herein represents an enantiomerically pure, a mixture of enantiomers (e.g. a racemic mixture), a mixture of diastereoisomers, a single regio-isomer and/or a mixture of regio-isomers, whenever and whatever applicable. A wiggle bond attached to a double bond indicates specifically that the configuration of the substituents on the double bond is undefined and the compound may thus be cis (Z), trans (E), or a mixture thereof.

As used herein, the singular forms “a”. “an” and “the” are intended to include the plural forms as well, unless the context clearly indicates otherwise. The term “and/or” includes any and all combinations of one or more of the associated listed items. It will be understood that the terms “comprises” and/or “comprising” specify the presence of stated features but do not preclude the presence or addition of one or more other features.

The invention may further be illustrated by the following non-limiting examples.

### Example 1-Synthesis of 2-(2-furylmethylene)malonic Acid

A reactor was charged with furfural (14.4 g) and malonic acid (15.02 g). The mixture was heated to 50° C. with stirring, then ammonium bicarbonate (1.19 g) was added. The reaction mixture was stirred at 50° C. for 1 hour then cooled to ambient temperature. Saturated sodium bicarbonate solution (100 mL) was charged, then the stirred mixture was acidified to pH 2 with 1 M aqueous hydrochloric acid to induce precipitation. The mixture was cooled to ˜5° C. and the formed solid was isolated by filtration, washing with minimal ice cold water. The resulting solid was dried in a vacuum oven (60° C.) to yield 12.8 g. 50%. Analysis by NMR confirmed the product to be 2-(2-furylmethylene) malonic acid.

### Example 2-Synthesis of dimethyl 2-(2-furylmethylene)malonate

A reactor was charged with furfural (14.4 g) and dimethyl malonate (24 g). The mixture was heated to 90° C. with stirring, then ammonium bicarbonate (1.19 g) was added. The reaction mixture was stirred at 90° C. for 3 hours then cooled to ambient temperature. The mixture was diluted with ethyl acetate (20 mL) and the organics washed with brine (20 mL). The organics were dried (Na₂SO₄), filtered and concentrated to an oil, which was purified by flash chromatography. Concentration of the appropriate fractions resulted in the precipitation of a solid which was dried in a vacuum oven (60° C.) to yield 19.5 g. 56%. Analysis by NMR confirmed the product to be dimethyl 2-(2-furylmethylene) malonate.

### Example 3

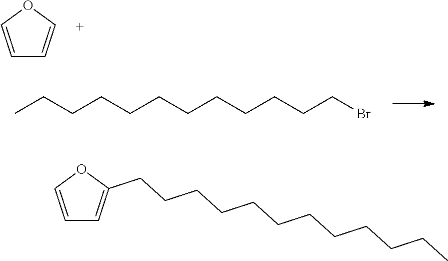

A reactor was charged with THF (140 mL) and this was cooled to 0° C. Furan (20 g) was charged followed by ⁿbutyl lithium (88.1 mL, 2.5M in hexanes), added dropwise over ˜15 minutes. Stirred at 0° C. for 15 minutes. A solution of dodecyl bromide (70.5 mL) in THF (60 mL) was added dropwise over ˜15 minutes. The cooling was removed and the mixture was warmed to 20° C. and stir for 2 hours. Concentrated to ˜25% of the original volume then cooled to 0° C. with stirring. Saturated aqueous ammonium bicarbonate solution (100 mL) was added dropwise over ˜30 minutes. The organics were then extracted twice with ethyl acetate (100 mL). The combined organic phases were dried (sodium sulfate), filtered and concentrated. The resulting liquid was purified over silica to yield a liquid, which was purified by vacuum distillation to yield a liquid. Analysis showed this to be reasonably pure 2-dodecylfuran (74% yield).

### Example 4

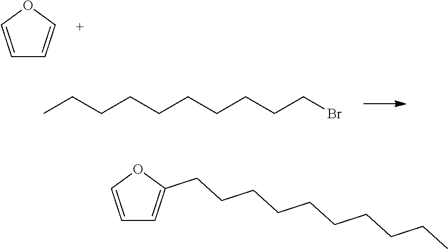

A reactor was charged with THF (60 mL) and this was cooled to 0° C. Furan (7.25 g) was charged followed by ⁿbutyl lithium (32.0 mL. 2.5M in hexanes), added dropwise over ˜15 minutes. Stirred at 0° C. for 15 minutes. A solution of 1-bromodecane (22.1 mL) in THF (40 mL) was added dropwise over ˜15 minutes. The cooling was removed and the mixture was warmed to 20° C. and stir for 2 hours. Concentrated to ˜25% of the original volume then cooled to 0° C. with stirring. Saturated aqueous ammonium bicarbonate solution (50 mL) was added dropwise over ˜30 minutes. The organics were then extracted twice with ethyl acetate (50 mL). The combined organic phases were dried (sodium sulfate), filtered and concentrated. The resulting liquid was purified over silica to yield a liquid, which was purified by vacuum distillation to yield a liquid. Analysis showed this to be reasonably pure 2-decylfuran (83% yield).

### Example 5

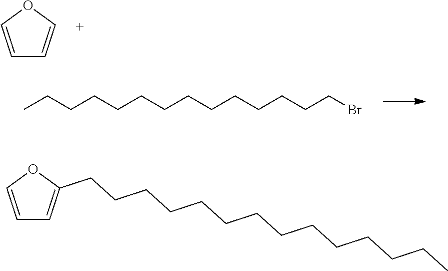

A reactor was charged with THF (60 mL) and this was cooled to 0° C. Furan (10.0 g) was charged followed by ⁿbutyl lithium (44.1 mL. 2.5M in hexanes), added dropwise over ˜15 minutes. Stirred at 0° C. for 15 minutes. A solution of 1-bromotetradecane (43.7 mL) in THF (40 mL) was added dropwise over ˜ 15 minutes. The cooling was removed and the mixture was warmed to 20° C. and stir for 2 hours. Concentrated to ˜25% of the original volume then cooled to 0° C. with stirring. Saturated aqueous ammonium bicarbonate solution (50 mL) was added dropwise over ˜30 minutes. The organics were then extracted twice with ethyl acetate (100 mL). The combined organic phases were dried (sodium sulfate), filtered and concentrated. The resulting liquid was purified over silica to yield a liquid, which was purified by vacuum distillation to yield a liquid. Analysis showed this to be reasonably pure 2-tetradecylfuran (68% yield).

### Example 6

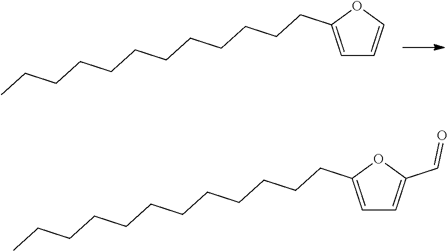 [SMILES: CCCCCCCCCCCCc1ccco1 ; CCCCCCCCCCCCc1ccc(C=O)o1]

To a reactor was charged DMF (15.5 mL) and 1,2-dichloroethane (60 mL) and this was cooled to 0° C. Phosphoryl chloride (16.8 mL) was added dropwise over ˜15 minutes. Stirred at 0° C. for 30 minutes. A solution of 2-dodecylfuran (27.81 g) in 1,2-dichloroethane (30 mL) was added dropwise over ˜30 minutes. The cooling was removed and the mixture was warmed to 20° C. and stir for 12 hours. Saturated aqueous sodium carbonate (150 mL) was added dropwise over ˜30 minutes. The organics were extracted twice with ethyl acetate (120 mL). The combined organic phases were dried (sodium sulfate), filtered and concentrated. The resulting liquid was purified by vacuum distillation to remove the volatiles. The residue was purified over silica, eluting with n-heptane. The fractions containing 5-dodecylfurfural were concentrated to yield a liquid. Analysis showed this to be reasonably pure 5-dodecylfurfural (58% yield).

### Example 7

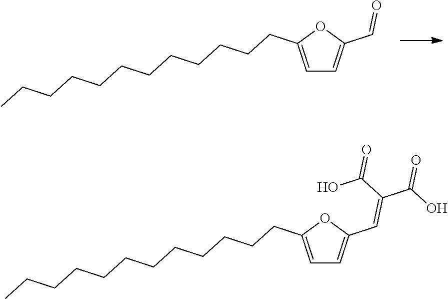 [SMILES: CCCCCCCCCCCCc1ccc(C=O)o1 ; CCCCCCCCCCCCc1ccc(C=C(C(=O)O)C(=O)O)o1]

To a reactor was charged 5-dodecylfurfural (5.5 g), malonic acid (1.84 g), ammonium bicarbonate (113 mg) and THF (25 mL). The mixture was heated to 50° C. with stirring and held for 20 hours. The THF was removed by distillation and mixture was cooled to 20° C. The mixture was washed twice with water (20 mL). The residue was taken up in THF (100 mL) and the mixture was dried (sodium sulfate), filtered and the solvent was removed by evaporation to yield a solid. Analysis showed this to be almost pure 2-[(5-dodecyl-2-furanyl)methylene]propanedioic acid (82% yield).

### Example 8

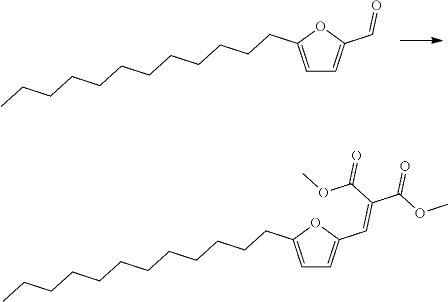 [SMILES: CCCCCCCCCCCCc1ccc(C=O)o1 ; CCCCCCCCCCCCc1ccc(C=C(C(=O)OC)C(=O)OC)o1]

To a reactor was charged 5-dodecylfurfural (11.2 g), dimethyl malonate (6.72 g) ammonium bicarbonate (333 mg) and 2-methyltetrahydrofuran (60 mL). The mixture was heated to 80° C. with stirring and held for 12 hours. The mixture was cooled to 20° C. and washed twice with saturated aqueous sodium chloride (50 mL). The organic phase was dried (sodium sulfate), filtered and the solvent and volatiles were removed by vacuum distillation. The residue was filtered over silica, eluting with n-heptane/ethyl acetate. The product containing fractions was concentrated to a liquid. Analysis showed this to be reasonably pure dimethyl 2-[(5-dodecyl-2-furanyl)methylene]propanedioate (79% yield).

### Example 9

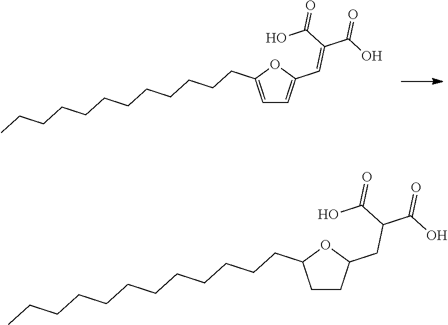 [SMILES: CCCCCCCCCCCCc1ccc(C=C(C(=O)O)C(=O)O)o1 ; CCCCCCCCCCCCC1CCC(CC(C(=O)O)C(=O)O)O1]

To a pressure reactor was charged the 2-[(5-dodecyl-2-furanyl)methylene]propanedioic acid (3.25 g), 5% Pd/C (20 mg) and methanol (30 ml). The reactor was sealed and flushed twice to 15 bar with nitrogen gas. The reactor was pressurized to 50 bar with hydrogen gas and the stirring was started immediately. When required, the hydrogen pressure was topped up to maintain a pressure of ˜50 bar. After 1 hour, the pressure was released and the reactor flushed twice to 15 bar with nitrogen gas. The reaction mixture was filtered to remove the catalyst and then concentrated to a solid. The material was purified over silica, eluting with n-heptane/ethyl acetate. The 2-[[5-(dodecyl)-2-furanyl]methyl]propanedioic acid containing fractions were concentrated to a solid. Analysis showed this to be almost pure 2-[[5-(dodecyl)-2-furanyl]methyl]propanedioic acid (89% yield).

### Example 10

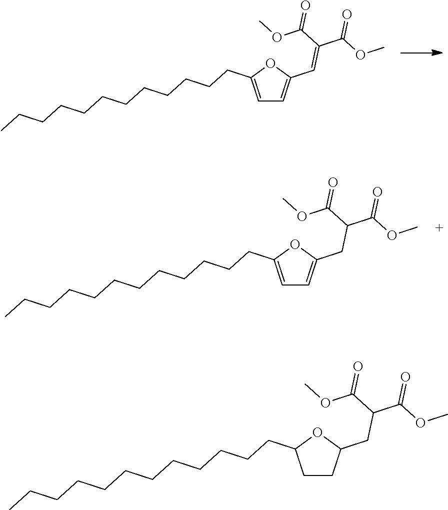

To a pressure reactor was charged dimethyl 2-[(5-dodecyl-2-furanyl)methylene]propanedioate (12.0 g). 5% Pd/C (60 mg) and methanol (50 mL). The reactor was sealed and flushed twice to 15 bar with nitrogen gas. The reactor was pressurized to 5 bar with hydrogen gas and the stirring was started immediately. When required, the hydrogen pressure was topped up to maintain a pressure of ˜5 bar. After 7.5 hours, the pressure was released and the reactor flushed twice to 15 bar with nitrogen gas. The reaction mixture was filtered to remove the catalyst and then concentrated to a liquid which analysis showed to be ˜2:1 ratio of the furanic product:tetrahydrofuranic product. The material was purified over silica, eluting with n-heptane/ethyl acetate. The dimethyl 2-[(5-dodecyl-2-furanyl)methyl]propanedioate containing fractions were concentrated to a liquid. Analysis showed this to be almost pure dimethyl 2-[(5-dodecyl-2-furanyl)methyl]propanedioate (44% yield). The dimethyl 2-[(tetrahydro-5-dodecyl-2-furanyl)methyl]propanedioate containing fractions were concentrated to a liquid. Analysis showed this to be reasonably pure 2-[(tetrahydro-5-dodecyl-2-furanyl)methyl]propanedioate (33% yield).

### Example 11

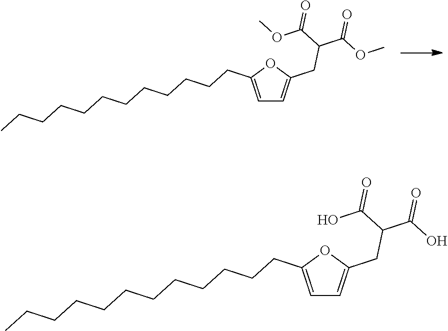

To a reactor was charged dimethyl 2-[(5-dodecyl-2-furanyl)methyl]propanedioate (5.0 g). 1M aqueous sulfuric acid (2.5 mL), and ethyl acetate (15 mL). Stirring was started and the mixture heated to 70° C. and held for 36 hours. The mixture was cooled to 20° C. and diluted in ethyl acetate (100 mL), then the solution was washed twice with water (25 mL) before being dried (sodium sulfate), filtered and partially concentrated. On cooling, a solid precipitated. This was isolated by filtration and washed with ethyl acetate before being dried in a vacuum oven. Analysis showed this to be reasonably pure 2-[[5-(dodecyl)-2-furanyl]methyl]propanedioic acid (58% yield).

### Example 12

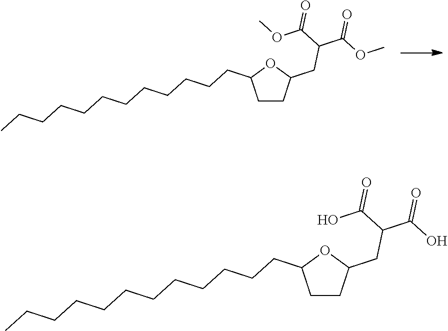

To a reactor was charged 2-[(tetrahydro-5-dodecyl-2-furanyl)methyl]propanedioate (3.9 g), 1M aqueous sulfuric acid (1.75 mL), and ethyl acetate (10 mL). Stirring was started and the mixture heated to 70° C. and held for 36 hours. The mixture was cooled to 20° C. and diluted in ethyl acetate (75 mL), then the solution was washed twice with water (20 mL) before being dried (sodium sulfate), filtered and partially concentrated. On cooling, a solid precipitated. This was isolated by filtration and washed with ethyl acetate before being dried in a vacuum oven. Analysis showed this to be reasonably pure 2-[[tetrahydro-5-(dodecyl)-2-furanyl]methyl]propanedioic acid (75% yield).

### Example 13

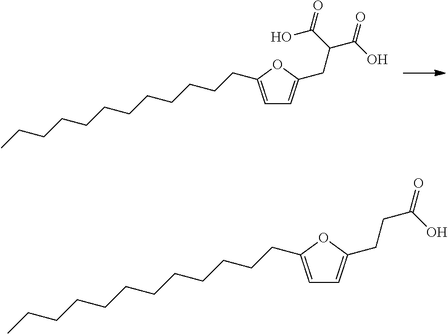 [SMILES: CCCCCCCCCCCCc1ccc(CC(C(=O)O)C(=O)O)o1 ; CCCCCCCCCCCCc1ccc(CCC(=O)O)o1]

To a reactor was charged 2-[[5-(dodecyl)-2-furanyl]methyl]propanedioic acid (2.1 g) and this was heated to 80° C. with stirring. When at temperature, 9M aqueous sulfuric acid was carefully added dropwise until no more gas evolution was observed. The pH was adjusted to ˜2 by addition of 1M sodium hydroxide. This was then washed twice with ethyl acetate (10 mL). The combined organics were dried (sodium sulfate), filtered and concentrated to yield a solid. This was filtered over silica, eluting with n-heptane/ethyl acetate. The desired product containing fractions were concentrated to a solid. Analysis showed this to be reasonably pure 5-(dodecyl)-2-furanpropanoic acid (29% yield).

### Example 14

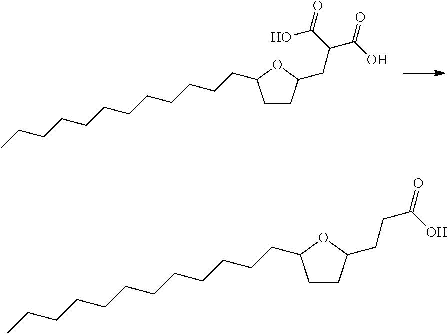 [SMILES: CCCCCCCCCCCCC1CCC(CC(C(=O)O)C(=O)O)O1 ; CCCCCCCCCCCCC1CCC(CCC(=O)O)O1]

To a reactor was charged 2-[[tetrahydro-5-(dodecyl)-2-furanyl]methyl]propanedioic acid (1.9 g) and this was heated to 80° C. with stirring. When at temperature, 9M aqueous sulfuric acid was carefully added dropwise until no more gas evolution was observed. The pH was adjusted to ˜2 by addition of 1M sodium hydroxide. This was then washed twice with ethyl acetate (10 mL). The combined organics were dried (sodium sulfate), filtered and concentrated to yield a solid. This was filtered over silica, eluting with n-heptane/ethyl acetate. The desired product containing fractions were concentrated to a solid. Analysis showed this to be reasonably pure 5-(dodecyl)-2-tetrahydrofuranpropanoic acid (67% yield).

### Example 15

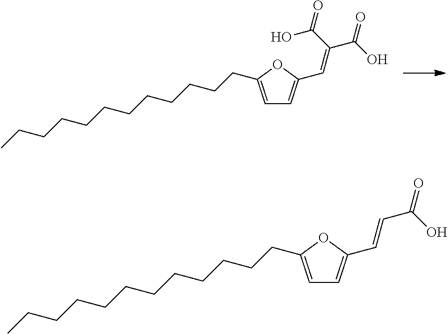 [SMILES: CCCCCCCCCCCCc1ccc(C=C(C(=O)O)C(=O)O)o1 ; CCCCCCCCCCCCc1ccc(/C=C/C(=O)O)o1]

To a reactor was charged 2-[(5-dodecyl-2-furanyl)methylene]propanedioic acid (2.3 g) and pyridine (15 mL) and the mixture was heated to 115° C. with stirring. After 5 hours, volatiles were removed by distillation. The mixture was cooled to 20° C. and then slowly acidified to PH ˜2 with 15% aqueous sulfuric acid. The organics were extracted with ethyl acetate (20 mL), then the organic phase was dried (sodium sulfate), filtered and concentrated to yield a solid. This was filtered over silica, eluting with n-heptane/ethyl acetate. The product containing fractions were concentrated to a solid. Analysis showed this to be reasonably pure 3-[5-(dodecyl)-2-furanyl]-2-propenoic acid (88% yield).

### Example 16

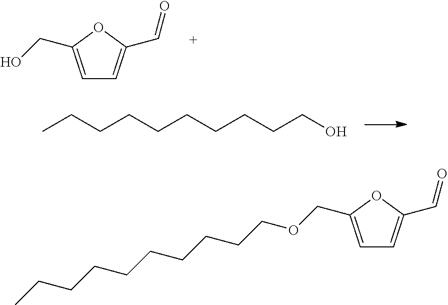

To a reactor was charged 1-decanol (90 mL), toluene (340 mL) and p-toluenesulfonic acid (280 mg) and the mixture was heated to 111° C. under Dean-Stark conditions. A mixture of HMF (20.0 g) in DCM (100 mL) was added over a period of 2 hours. The mixture was refluxed for a further 1 hour then cooled to 20° C. The mixture was washed with saturated aqueous sodium bicarbonate (50 mL). The organic phase was dried (sodium sulfate), filtered and partially concentrated. On cooling, a solid formed (di-HMF ether) and this was removed by filtration. The filtrate was then purified by distillation to remove the lower boiling fractions. The residue was filtered over silica, eluting with n-heptane. The product containing fractions was concentrated to a liquid. Analysis showed this to be almost pure 5-(decoxymethyl)furfural (41% yield).

### Example 17

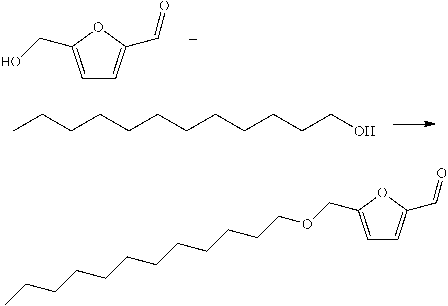

To a reactor was charged 1-dodecanol (4.5 mL), toluene (17 mL) and p-toluenesulfonic acid (14 mg) and the mixture was heated to 111° C. under Dean-Stark conditions. A mixture of HMF (1.0 g) in DCM (5 mL) was added over a period of 2 hours. The mixture was refluxed for a further 1 hour then cooled to 20° C. The mixture was washed with saturated aqueous sodium bicarbonate (50 mL). The organic phase was dried (sodium sulfate), filtered and partially concentrated. On cooling, a solid formed (di-HMF ether) and this was removed by filtration. The filtrate was then purified by distillation to remove the lower boiling fractions. The residue was filtered over silica, eluting with n-heptane. The product containing fractions was concentrated to a liquid. Analysis showed this to be almost pure 5-(dodecoxymethyl)furfural (67% yield).

### Example 18

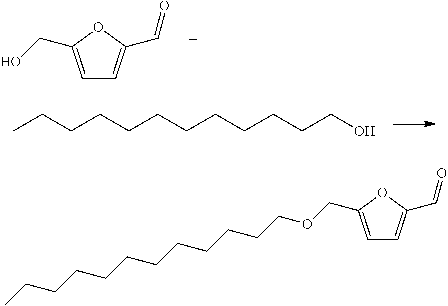

To a reactor was charged 1-tetradecanol (4.5 mL), toluene (17 mL) and p-toluenesulfonic acid (14 mg) and the mixture was heated to 111° C. under Dean-Stark conditions. A mixture of HMF (1.0 g) in DCM (5 mL) was added over a period of 2 hours. The mixture was refluxed for a further 1 hour then cooled to 20° C. The mixture was washed with saturated aqueous sodium bicarbonate (50 mL). The organic phase was dried (sodium sulfate), filtered and partially concentrated. On cooling, a solid formed (di-HMF ether) and this was removed by filtration. The filtrate was then purified by distillation to remove the lower boiling fractions. The residue was filtered over silica, eluting with n-heptane. The product containing fractions was concentrated to a liquid. Analysis showed this to be almost pure 5-(tetradecoxymethyl) furfural (62% yield).

### Example 19

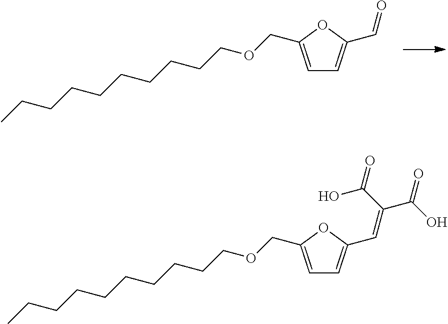 [SMILES: CCCCCCCCCCOCc1ccc(C=O)o1 ; CCCCCCCCCCOCc1ccc(C=C(C(=O)O)C(=O)O)o1]

To a reactor was charged 5-(decoxymethyl) furfural (4.0 g), malonic acid (1.43 g), ammonium bicarbonate (113 mg) and THF (20 mL). The mixture was heated to 50° C. with stirring and held for 20 hours. The THF was removed by distillation and mixture was cooled to 20° C. The mixture was washed twice with water (20 mL). The residue was taken up in THF (20 mL) and the mixture was dried (sodium sulfate), filtered and the solvent was removed by evaporation to yield a solid. Analysis showed this to be almost pure 2-[(5-decoxymethyl-2-furanyl)methylene]propanedioic acid (93% yield).

### Example 20

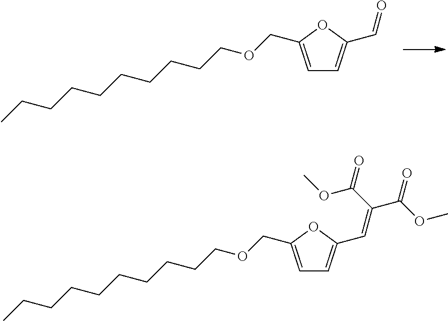 [SMILES: CCCCCCCCCCOCc1ccc(C=O)o1 ; CCCCCCCCCCOCc1ccc(C=C(C(=O)OC)C(=O)OC)o1]

To a reactor was charged 5-(decoxymethyl) furfural (4.0 g), dimethyl malonate (2.40 g) ammonium bicarbonate (119 mg) and 2-methyltetrahydrofuran (20 mL). The mixture was heated to 80° C. with stirring and held for 12 hours. The mixture was cooled to 20° C. and washed twice with saturated aqueous sodium chloride (20 mL). The organic phase was dried (sodium sulfate), filtered and the solvent and volatiles were removed by vacuum distillation. The residue was filtered over silica, eluting with n-heptane/ethyl acetate. The product containing fractions was concentrated to a liquid. Analysis showed this to be reasonably pure dimethyl 2-[(5-decoxymethyl-2-furanyl)methylene]propanedioate (71% yield).

### Example 21

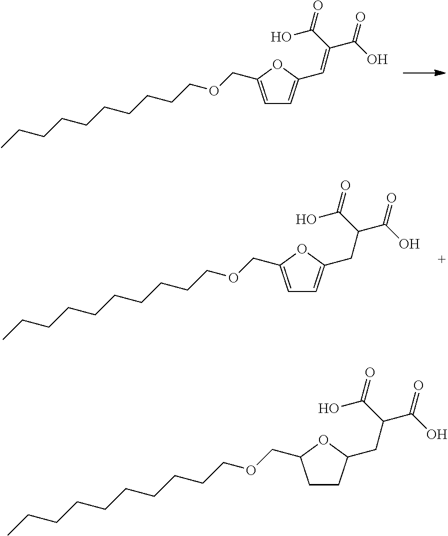 [SMILES: CCCCCCCCCCOCc1ccc(C=C(C(=O)O)C(=O)O)o1 ; CCCCCCCCCCOCc1ccc(CC(C(=O)O)C(=O)O)o1 ; CCCCCCCCCCOCC1CCC(CC(C(=O)O)C(=O)O)O1]

To a pressure reactor was charged the 2-[(5-decoxymethyl-2-furanyl)methylene]propanedioic acid (1.75 g). 5% Pd/C (11 mg) and methanol (25 ml). The reactor was sealed and flushed twice to 15 bar with nitrogen gas. The reactor was pressurized to 5 bar with hydrogen gas and the stirring was started immediately. When required, the hydrogen pressure was topped up to maintain a pressure of ˜5 bar. After 1 hour, the pressure was released and the reactor flushed twice to 15 bar with nitrogen gas. The reaction mixture was filtered to remove the catalyst and then concentrated to a liquid which analysis showed to be ˜1:1 ratio of the furanic product:tetrahydrofuranic product. The material was purified over silica, eluting with n-heptane/ethyl acetate. The 2-[[5-(decoxymethyl)-2-furanyl]methyl]propanedioic acid and 2-[[5-(decoxymethyl)-2-furanyl]methyl]propanedioic acid containing fractions were concentrated to a liquid. Analysis showed this to be almost pure mixture of the two di-acid products in ˜1:1 ratio (87% yield).

### Example 22

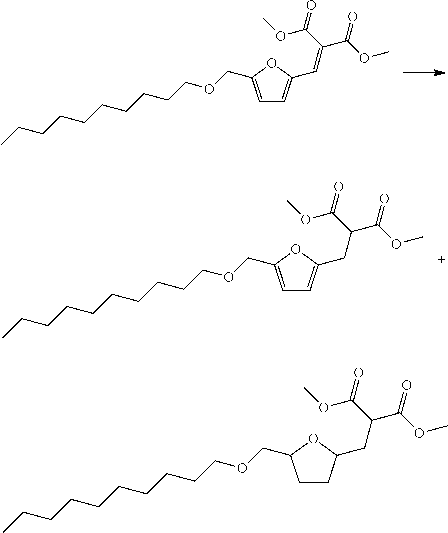

To a pressure reactor was charged dimethyl 2-[(5-decoxymethyl-2-furanyl)methylene]propanedioate (1.82 g). 5% Pd/C (11 mg) and methanol (25 mL). The reactor was sealed and flushed twice to 15 bar with nitrogen gas. The reactor was pressurized to 5 bar with hydrogen gas and the stirring was started immediately. When required, the hydrogen pressure was topped up to maintain a pressure of ˜5 bar. After 7.5 hours, the pressure was released and the reactor flushed twice to 15 bar with nitrogen gas. The reaction mixture was filtered to remove the catalyst and then concentrated to a liquid which analysis showed to be ˜3:1 ratio of the furanic product:tetrahydrofuranic product. The material was purified over silica, eluting with n-heptane/ethyl acetate. The dimethyl 2-[(5-decoxymethyl-2-furanyl)methyl]propanedioate containing fractions were concentrated to a liquid. Analysis showed this to be almost pure dimethyl 2-[(5-decoxymethyl-2-furanyl)methyl]propanedioate (59% yield). The dimethyl 2-[(tetrahydro-5-decoxymethyl-2-furanyl)methyl]propanedioate containing fractions were concentrated to a liquid. Analysis showed this to be reasonably pure 2-[(tetrahydro-5-decoxymethyl-2-furanyl)methyl]propanedioate (18% yield).

1. A method for the preparation of a surfactant, said method comprising reacting a furanic compound according to formula (V), or a salt thereof, with a malonate according to formula (VI), or a salt thereof, to obtain a first furan-based surfactant precursor of formula (I) or a salt thereof, 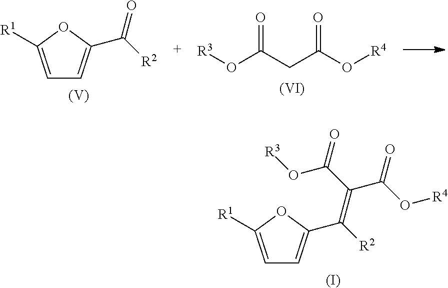
wherein R¹ is H or CH₂OX, wherein X is H or an aliphatic group;
wherein R², R³ and R⁴ are independently H or an aliphatic group; and
wherein the method further comprises one or more steps to convert the first furan-based surfactant precursor of formula (I), or a salt thereof, to a surfactant, which surfactant comprises:
a) a furan moiety comprising a backbone structure of formula (VII), 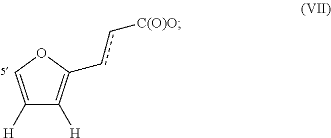
b) a hydrophilic moiety, and
c) a hydrophobic moiety comprising an aliphatic chain comprising at least 6 carbon atoms.

2. The method according to claim 1, wherein the one or more steps to convert the first furan-based surfactant precursor of formula (I), or a salt thereof, to a surfactant comprise a hydrogenation, decarboxylation, or 5′-position manipulation, hydrogenation followed by α-substitution or a combination thereof.

3. The method according to claim 1, wherein the one or more steps to convert the first furan-based surfactant precursor of formula (I), or a salt thereof, to a surfactant comprise reacting the first furan-based surfactant precursor of formula (I), or a salt thereof, to obtain a second-level compound of formula (IIa), or a salt thereof, comprising hydrogenation; 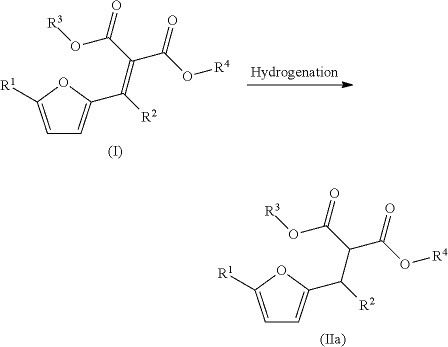
wherein for formula (I): R¹, R², R³ and R⁴ are as defined for formula (I) in claim 1; and
wherein the second-level compound of formula (IIa), or a salt thereof, is continued in one or more steps to convert the second-level compound of formula (IIa), or a salt thereof, to said surfactant, in which case for formula (IIa): R¹, R², R³ and R⁴ are as defined for formula (I) in claim 1; or
wherein the second-level compound of formula (IIa), or a salt thereof, is said surfactant in which case for formula (IIa), or a salt thereof, R¹, R², R³ and R⁴ are as defined for formula (I) in claim 1, provided that that at least one of R¹-R³ is an aliphatic chain comprising more than 6 carbon atoms and R⁴ is H.

4. The method according to claim 1, wherein the one or more steps to convert the first furan-based surfactant precursor of formula (I), or a salt thereof, to a surfactant comprise reacting the first furan-based surfactant precursor of formula (I), or a salt thereof, to obtain a second-level compound of formula (IIb), or a salt thereof, comprising decarboxylation; 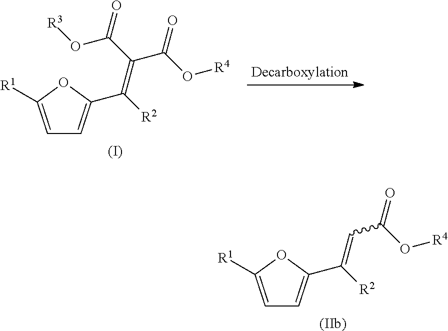
wherein for formula (I): R¹, R², R³ and R⁴ are as defined for formula (I) in claim 1; and
wherein the second-level compound of formula (IIb), or a salt thereof, is continued in one or more steps to convert the second-level compound of formula (IIb), or a salt thereof, to said surfactant, in which case for formula (IIb): R¹, R² and R⁴ are as defined for formula (I) in claim 1; or
wherein the second-level compound of formula (IIb), or a salt thereof, is said surfactant in which case for formula (IIb) R¹ and R² are as defined for formula (I) in claim 1 provided that at least one of R¹ and R² is an aliphatic chain comprising more than 6 carbon atoms and R⁴ is H.

5. The method according to claim 1, wherein the one or more steps to convert the first furan-based surfactant precursor of formula (I), or a salt thereof, to a surfactant comprise reacting the first furan-based surfactant precursor of formula (I), or a salt thereof, wherein R¹ is H or CH₂OH, to obtain a second-level compound of formula (IIc), or a salt thereof, comprising a substitution on the furan with a reactant to introduce hydrophilic group G; 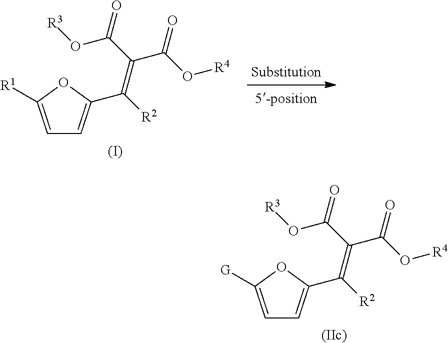
wherein G is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium, methylene sulfate, methylene sulfonate, methylene sulfinate, methylene thiosulfate, methylene sulfamidate, methylene carboxylate, methylene sarcosinate and taurate, methylene phosphate, methylene pyrophosphate, methylene phosphonate, methylene amines or ammonium, methylene polyammonium, methylene hydroxyammonium, methylene pyridinium, methylene picolinium, methylene imidazolinium, methylene benzimidazolinium, methylene oxonium, methylene sulfonium, and methylene phosphonium, poly(ethylene oxide), poly(co-ethylene oxide co-propylene oxide) (also referred to as poloxamers), polyglycosides, isosorbide and its derivatives, 1,4-sorbitane and its derivatives;
wherein for formula (I): R², R³ and R⁴ are as defined for formula (I) in claim 1; and
wherein the second-level compound of formula (IIc), or a salt thereof, is continued in one or more steps to convert the second-level compound of formula (IIc), or a salt thereof, to said surfactant in which case for formula (IIc): R², R³ and R⁴ are as defined for formula (I) in claim 1; or
wherein the second-level compound of formula (IIc), or a salt thereof, is said surfactant in which case for formula (IIc) R², R³ and R⁴ are as defined for formula (I) in claim 1 provided that at least one of R², R³ and R⁴ is an aliphatic chain comprising more than 6 carbon atoms.

6. The method according to claim 1, wherein the one or more steps to convert the first furan-based surfactant precursor of formula (I), or a salt thereof, to a surfactant comprise reacting the first furan-based surfactant precursor of formula (I), or a salt thereof, wherein R¹ is H to obtain a second-level compound of formula (IId), or a salt thereof, comprising substitution on the furan with a reactant to introduce hydrophobic group A; 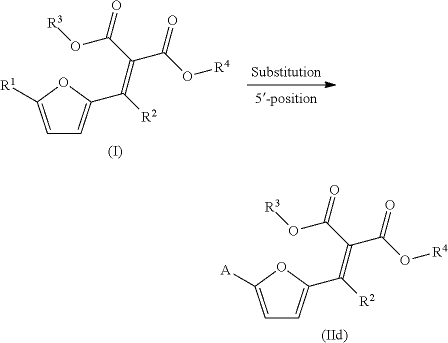
wherein A is an aliphatic chain comprising at least six carbon atoms,
wherein for formula (I): R², R³ and R⁴ are as defined for formula (I) in claim 1; and
wherein the second-level compound of formula (IId), or a salt thereof, is continued in one or more steps to convert the second-level compound of formula (IId), or a salt thereof, to said surfactant in which case for formula (IId): R², R³ and R⁴ are as defined for formula (I) in claim 1; or
wherein the second-level compound of formula (IId), or a salt thereof, is said surfactant in which case for formula (IId) R², R³ and R⁴ are as defined for formula (I) in claim 1 provided that at least one of R³ and R⁴ is H.

7. The method according to claim 3, wherein the one or more steps to convert the second-level compound, or a salt thereof, of formula (IIa) to a surfactant comprise reacting the second-level compound, or a salt thereof, of formula (IIa) wherein R¹ is H or CH₂OH in a reaction comprising a substitution on the furan with a reactant to introduce a hydrophobic group A or a hydrophilic group G, to obtain a third-level compound, or a salt thereof, of any of formulae (IIIb) and (IIIc) 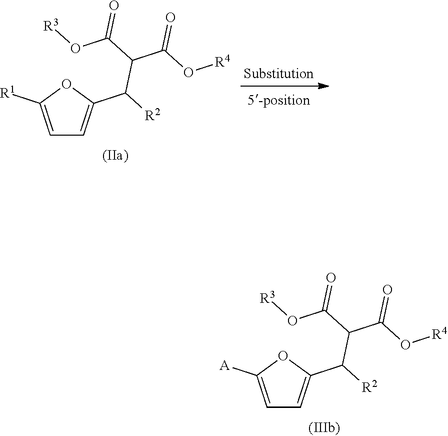 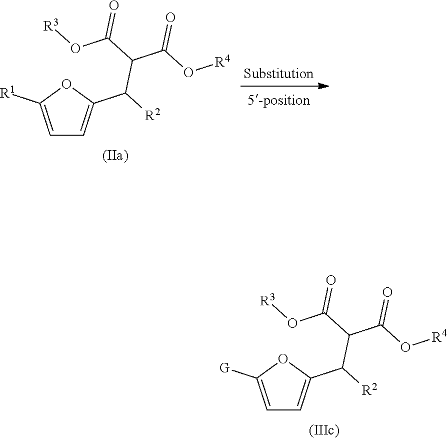
wherein A is an aliphatic chain comprising at least six carbon atoms;
wherein G is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium, methylene sulfate, methylene sulfonate, methylene sulfinate, methylene thiosulfate, methylene sulfamidate, methylene carboxylate, methylene sarcosinate and taurate, methylene phosphate, methylene pyrophosphate, methylene phosphonate, methylene amines or ammonium, methylene polyammonium, methylene hydroxyammonium, methylene pyridinium, methylene picolinium, methylene imidazolinium, methylene benzimidazolinium, methylene oxonium, methylene sulfonium, and methylene phosphonium, poly(ethylene oxide), poly(co-ethylene oxide co-propylene oxide) (also referred to as poloxamers), polyglycosides, isosorbide and its derivatives, 1,4-sorbitane and its derivatives;
wherein the third-level compound, or a salt thereof, of any of formulae (IIIb) and (IIIc) is continued in one or more steps to convert said third-level compound, or a salt thereof, to said surfactant in which case for formulae (IIIb) and (IIIc): R², R³ and R⁴ are R², R³ and R⁴ are independently H or an aliphatic group; or
wherein the third-level compound, or a salt thereof, of any of formulae (IIIb) and (IIIc) is said surfactant in which case
for formula (IIIb): R², R³ and R⁴ are independently H or an aliphatic group, provided that least R⁴ is H;
for formula (IIIc): R², R³ and R⁴ are independently H or an aliphatic group, provided that at least one of R², R³ and R⁴ is an aliphatic chain comprising six or more carbon atoms.

8. The method according to claim 3, wherein the one or more steps to convert the second-level compound, or a salt thereof, of formula (IIa) to a surfactant comprise reacting the second-level compound, or a salt thereof, of formula (IIa) in a reaction comprising an α-substitution with a reaction comprising R⁵ to obtain a third-level compound, or a salt thereof, of formula (IIIa), optionally followed by reacting the third-level compound, or a salt thereof, of formula (IIIa) in a decarboxylation reaction to obtain the surfactant of formula (IVc), 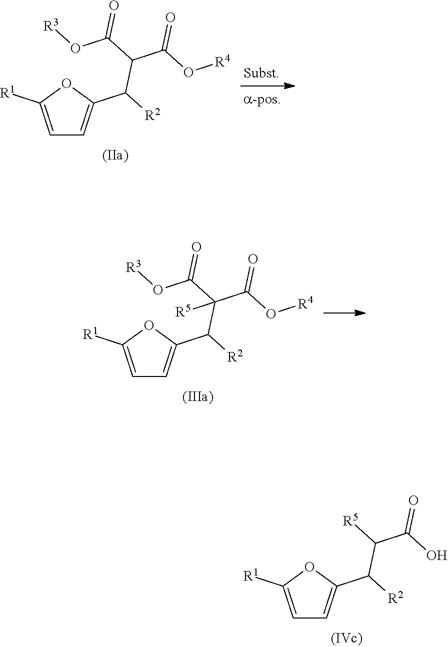
wherein R⁵ is an aliphatic chain comprising at least six carbon atoms;
wherein for formula (IIa) and (IVc): R¹ is H or CH₂OX, wherein X is H or an aliphatic group and R², R³ and R⁴ are independently H or an aliphatic group; and
wherein for formula (IIIa) R¹ is H or CH₂OX, wherein X is H or an aliphatic group and R², R³ and R⁴ are independently H or an aliphatic group if the method comprises said optional decarboxylation reaction; or
wherein the third-level compound, or a salt thereof, of formula (IIIa) is said surfactant in which case for formula (IIIa): R¹ is H or CH₂OX, wherein X is H or an aliphatic group R², R³ and R⁴ are independently H or an aliphatic group, provided that at least one of R¹, R², R³ and R⁵ is an aliphatic chain comprising six or more carbon atoms and R⁴ is H.

9. The method according to claim 4, said method comprising continuing the second-level compound, or a salt thereof, of formula (IIb) in one or more steps to convert said second-level compound, or a salt thereof, to the surfactant, wherein said one or more steps comprise reacting said second-level compound, or a salt thereof, in a reaction comprising a hydrogenation to obtain a third-level compound, or a salt thereof, of any of formulae (IIIe) 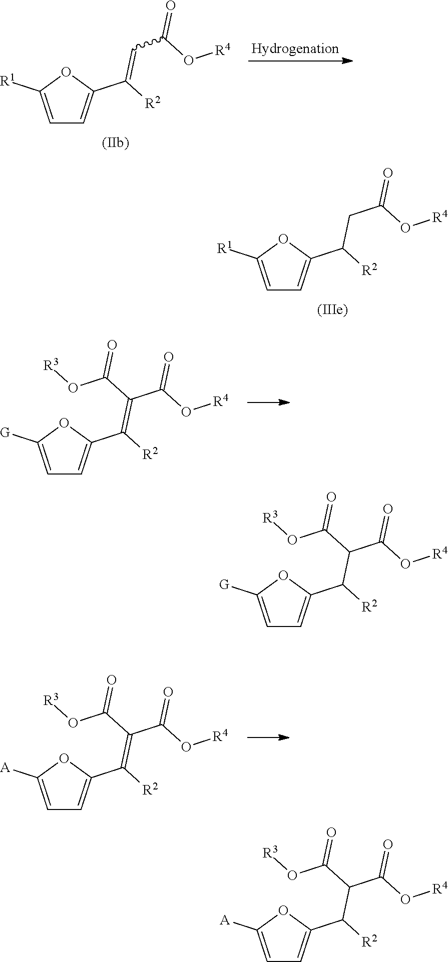
wherein the third-level compound, or a salt thereof, of formula (IIIe) is continued in one or more steps to convert said third-level compound, or a salt thereof, to said surfactant in which case for formula (Ille): R¹ is H or CH₂OX, wherein X is H or an aliphatic group, and R², R³ and R⁴ are independently H or an aliphatic group; or
wherein the third-level compound, or a salt thereof, of formula (IIIe) is said surfactant in which case for formula (IIIe): R¹ is H or CH₂OX, wherein X is H or an aliphatic group, and R², and R⁴ are independently H or an aliphatic group, provided that at least one of R¹ and R² is an aliphatic chain comprising six or more carbon atoms and R⁴ is H.

10. The method according to claim 3, said method comprising continuing the second-level compound, or a salt thereof, of formula (IIa) in one or more steps to convert said second-level compound, or a salt thereof, to the surfactant, wherein said one or more steps comprise reacting said second-level compound, or a salt thereof, in a reaction comprising a decarboxylation to obtain a third-level compound, or a salt thereof, of formula (Ille) 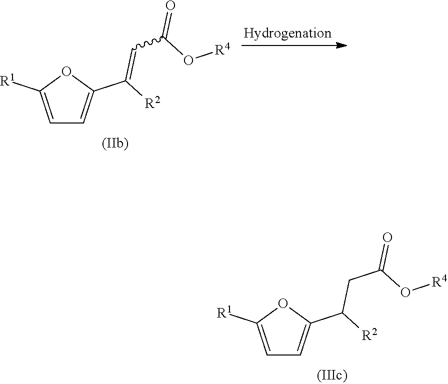
wherein the third-level compound, or a salt thereof, of formula (IIIe) is continued in one or more steps to convert said third-level compound, or a salt thereof, in which case for formula (IIIe): R¹, is H or CH₂OX, wherein X is H or an aliphatic group. and R² and R⁴ are independently H or an aliphatic group; or
wherein the third-level compound, or a salt thereof, of formula (IIIe) is said surfactant in which case for formula (IIIe): R¹, is H or CH₂OX, wherein X is H or an aliphatic group, and R² and R⁴ are independently H or an aliphatic group provided that at least one of R¹ and R² is an aliphatic chain comprising six or more carbon atoms and R⁴ is H.

11. The method according to claim 9, said method comprising continuing the third-level compound, or a salt thereof, of formula (IIIe) in one or more steps to convert said third-level compound, or a salt thereof, to the surfactant, wherein said one or more steps comprise reacting said third-level compound, or a salt thereof, in a reaction comprising a substitution on the furan with a reactant comprising a hydrophobic group A, a substitution on the furan with a reactant comprising a hydrophobic group G or an α-substitution with a reaction comprising R⁵ to obtain the surfactant of formula (IVa) or (IVc), respectively 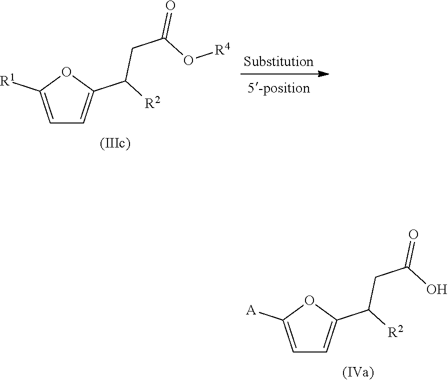 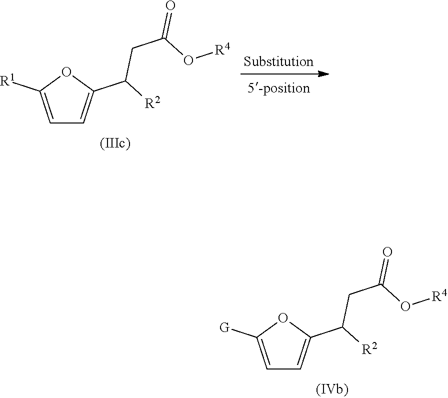 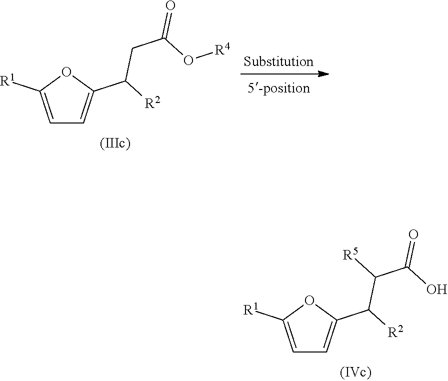
wherein for formula (IVa): A is an aliphatic chain comprising at least six carbon atoms and R² is H or an aliphatic group,
wherein for formula (IVb): G is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium, methylene sulfate, methylene sulfonate, methylene sulfinate, methylene thiosulfate, methylene sulfamidate, methylene carboxylate, methylene sarcosinate and taurate, methylene phosphate, methylene pyrophosphate, methylene phosphonate, methylene amines or ammonium, methylene polyammonium, methylene hydroxyammonium, methylene pyridinium, methylene picolinium, methylene imidazolinium, methylene benzimidazolinium, methylene oxonium, methylene sulfonium, and methylene phosphonium, poly(ethylene oxide), poly(co-ethylene oxide co-propylene oxide) (also referred to as poloxamers), polyglycosides, isosorbide and its derivatives, 1,4-sorbitane and its derivatives, and R² and R⁴ are independently H or an aliphatic group provided that at least one of R² and R⁴ is an aliphatic chain comprising more than 6 carbon atoms, and
wherein for formula (IVc): R⁵ is an aliphatic group and R¹ is H or CH₂OX, wherein X is H or an aliphatic group, and R² is H or an aliphatic group.

12. The method according to claim 7, said method comprising continuing the third-level compound, or a salt thereof, of any of formula (IIIb) in one or more steps to convert said third-level compound, or a salt thereof, to the surfactant, wherein said one or more steps comprise reacting said third-level compound, or a salt thereof, in a reaction comprising a decarboxylation to obtain the surfactant of formula (IVa) 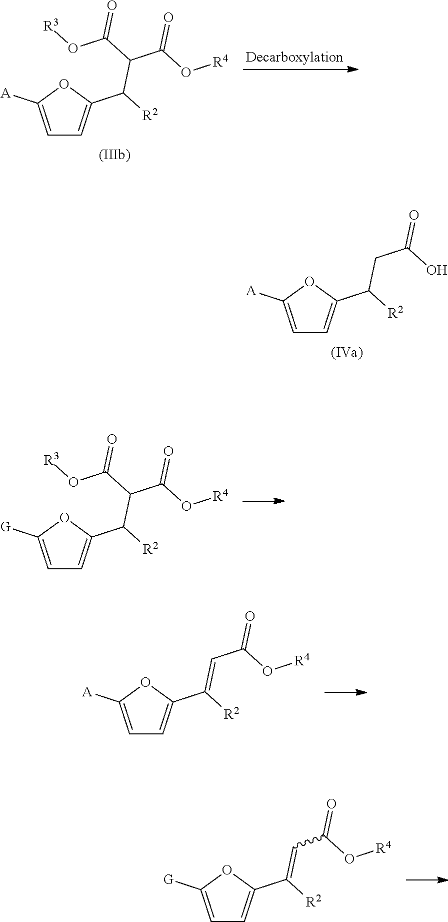
wherein A is an aliphatic chain comprising at least six carbon atoms and wherein for formula (IVa) R² is H or an aliphatic group.

13. The method according to claim 1, wherein for all formulae, R¹ and R² are H.

14. The method according to claim 1, wherein for all formulae, R³ and R⁴ are H, and wherein R¹ is CH₂OX wherein X is an aliphatic chain and wherein R² is H.

15. The method according to claim 1, wherein for all formulae, R² is an aliphatic chain.

16. The method according to claim 1, wherein the hydrophilic moiety is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium and non-ionic groups.

17. The method according to claim 4, wherein the one or more steps to convert the second-level compound, or a salt thereof, of formula (IIa) or (IIb) to a surfactant comprise reacting the second-level compound, or a salt thereof, of formula (IIa) or (IIb) wherein R¹ is H or CH₂OH in a reaction comprising a substitution on the furan with a reactant to introduce a hydrophobic group A or a hydrophilic group G, to obtain a third-level compound, or a salt thereof, of any of formulae (IIId) and (IIIf) 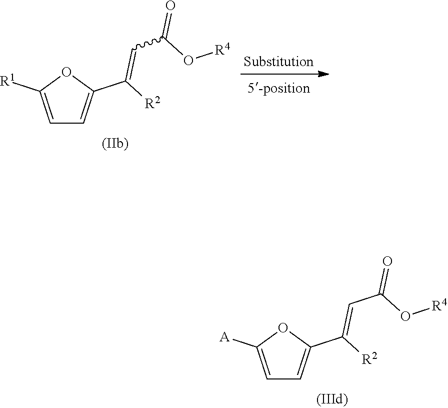 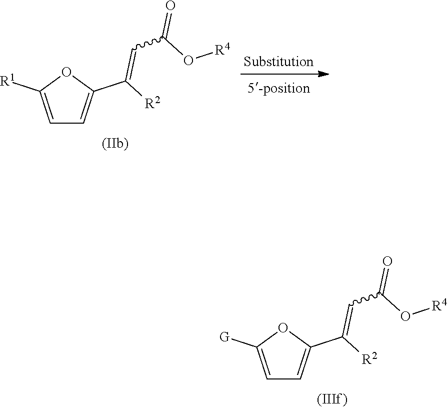
wherein A is an aliphatic chain comprising at least six carbon atoms;
wherein G is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium, methylene sulfate, methylene sulfonate, methylene sulfinate, methylene thiosulfate, methylene sulfamidate, methylene carboxylate, methylene sarcosinate and taurate, methylene phosphate, methylene pyrophosphate, methylene phosphonate, methylene amines or ammonium, methylene polyammonium, methylene hydroxyammonium, methylene pyridinium, methylene picolinium, methylene imidazolinium, methylene benzimidazolinium, methylene oxonium, methylene sulfonium, and methylene phosphonium, poly(ethylene oxide), poly(co-ethylene oxide co-propylene oxide) (also referred to as poloxamers), polyglycosides, isosorbide and its derivatives, 1,4-sorbitane and its derivatives;
wherein the third-level compound, or a salt thereof, of any of formulae (IIId) and (IIIf) is continued in one or more steps to convert said third-level compound, or a salt thereof, to said surfactant in which case for formulae (IIId) and (IIIf): R², R³ and R⁴ are independently H or an aliphatic group; or
wherein the third-level compound, or a salt thereof, of any of formulae (IIId) and (IIIf) is said surfactant in which case
for formula (IIId): R² is H or an aliphatic group, and R⁴ is H;
for formula (IIIf) R² and R⁴ are independently H or an aliphatic group, provided that at least one of R² and R⁴ is an aliphatic chain comprising six or more carbon atoms.

18. The method according to claim 5, said method comprising continuing the second-level compound, or a salt thereof, of formula (IIc) in one or more steps to convert said second-level compound, or a salt thereof, to the surfactant, wherein said one or more steps comprise reacting said second-level compound, or a salt thereof, in a reaction comprising a hydrogenation to obtain a third-level compound, or a salt thereof, of formula (IIIc) 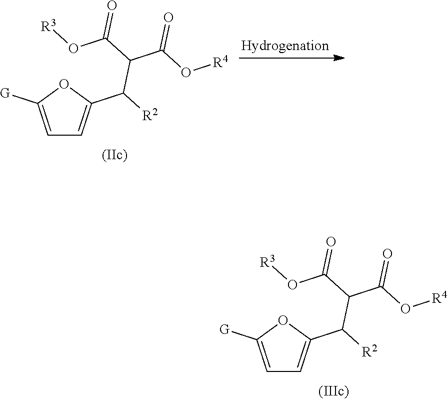
wherein G is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium, methylene sulfate, methylene sulfonate, methylene sulfinate, methylene thiosulfate, methylene sulfamidate, methylene carboxylate, methylene sarcosinate and taurate, methylene phosphate, methylene pyrophosphate, methylene phosphonate, methylene amines or ammonium, methylene polyammonium, methylene hydroxyammonium, methylene pyridinium, methylene picolinium, methylene imidazolinium, methylene benzimidazolinium, methylene oxonium, methylene sulfonium, and methylene phosphonium, poly(ethylene oxide), poly(co-ethylene oxide co-propylene oxide) (also referred to as poloxamers), polyglycosides, isosorbide and its derivatives, 1,4-sorbitane and its derivatives;
wherein the third-level compound, or a salt thereof, of formula (IIIc) is continued in one or more steps to convert said third-level compound, or a salt thereof, to said surfactant in which case for formula (IIIc): R¹ is H or CH₂OX, wherein X is H or an aliphatic group, and R², R³ and R⁴ are independently H or an aliphatic group; or
wherein the third-level compound, or a salt thereof, of formula (IIIc) is said surfactant in which case R², R³ and R⁴ are independently H or an aliphatic group, provided that at least one of R², R³ and R⁴ is an aliphatic chain comprising six or more carbon atoms.

19. The method according to claim 6, said method comprising continuing the second-level compound, or a salt thereof, of formula (IId) in one or more steps to convert said second-level compound, or a salt thereof, to the surfactant, wherein said one or more steps comprise reacting said second-level compound, or a salt thereof, in a reaction comprising a hydrogenation to obtain a third-level compound, or a salt thereof, of formula (IIIb) 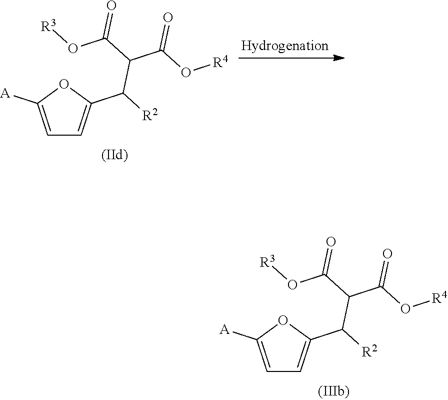
wherein A is an aliphatic chain comprising at least six carbon atoms;
wherein the third-level compound, or a salt thereof, of formula (IIIb) is continued in one or more steps to convert said third-level compound, or a salt thereof, to said surfactant in which case for formula (IIIb): R², R³ and R⁴ are independently H or an aliphatic group; or
wherein the third-level compound, or a salt thereof, of formula (IIIb) is said surfactant in which case R², R³ and R⁴ are independently H or an aliphatic group, provided that at least R⁴ is H.

20. The method according to claim 5, said method comprising continuing the second-level compound, or a salt thereof, of formula (IIc) in one or more steps to convert said second-level compound, or a salt thereof, to the surfactant, wherein said one or more steps comprise reacting said second-level compound, or a salt thereof, in a reaction comprising a decarboxylation to obtain a third-level compound, or a salt thereof, of formula (IIIf) 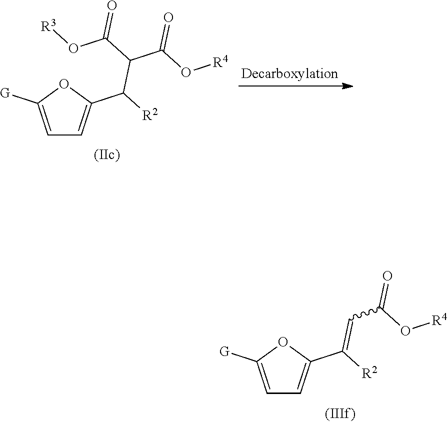
wherein G is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium, methylene sulfate, methylene sulfonate, methylene sulfinate, methylene thiosulfate, methylene sulfamidate, methylene carboxylate, methylene sarcosinate and taurate, methylene phosphate, methylene pyrophosphate, methylene phosphonate, methylene amines or ammonium, methylene polyammonium, methylene hydroxyammonium, methylene pyridinium, methylene picolinium, methylene imidazolinium, methylene benzimidazolinium, methylene oxonium, methylene sulfonium, and methylene phosphonium, poly(ethylene oxide), poly(co-ethylene oxide co-propylene oxide) (also referred to as poloxamers), polyglycosides, isosorbide and its derivatives, 1,4-sorbitane and its derivatives;
wherein the third-level compound, or a salt thereof, of formula (IIIf) is continued in one or more steps to convert said third-level compound, or a salt thereof, in which case for formula (IIIf) R¹, is H or CH₂OX, wherein X is H or an aliphatic group, and R² and R⁴ are independently H or an aliphatic group; or
wherein the third-level compound, or a salt thereof, of formula (IIIf) is said surfactant in which case for formula (IIIf): R² and R⁴ R² and R⁴ are independently H or an aliphatic group, provided that at least one of R¹ and R² is an aliphatic chain comprising six or more carbon atoms.

21. The method according to claim 6, said method comprising continuing the second-level compound, or a salt thereof, of formula (IId) in one or more steps to convert said second-level compound, or a salt thereof, to the surfactant, wherein said one or more steps comprise reacting said second-level compound, or a salt thereof, in a reaction comprising a decarboxylation to obtain a third-level compound, or a salt thereof, of formula (IIId) 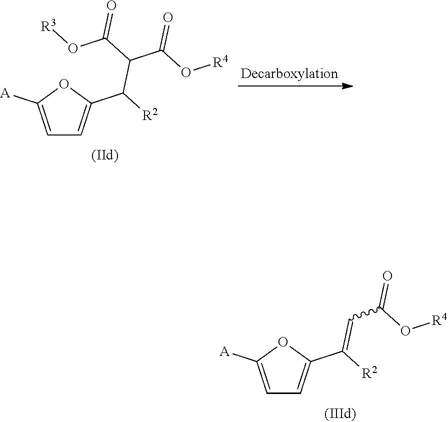
wherein A is an aliphatic chain comprising at least six carbon atoms,
wherein the third-level compound, or a salt thereof, of any of formula (IIId) is continued in one or more steps to convert said third-level compound, or a salt thereof, formula to said surfactant in which case for formula (IIId): R², and R⁴ are independently H or an aliphatic group; or
wherein the third-level compound, or a salt thereof, of any of formula (IIId) is said surfactant in which case for formula for formula (IIId): R² is H or an aliphatic group, and R⁴ is H.

22. The method according to claim 10, said method comprising continuing the third-level compound, or a salt thereof, of formula (IIIe) in one or more steps to convert said third-level compound, or a salt thereof, to the surfactant, wherein said one or more steps comprise reacting said third-level compound, or a salt thereof, in a reaction comprising a substitution on the furan with a reactant comprising a hydrophobic group A, a substitution on the furan with a reactant comprising a hydrophobic group G or an α-substitution with a reaction comprising R⁵ to obtain the surfactant of formula (IVa) or (IVc), respectively 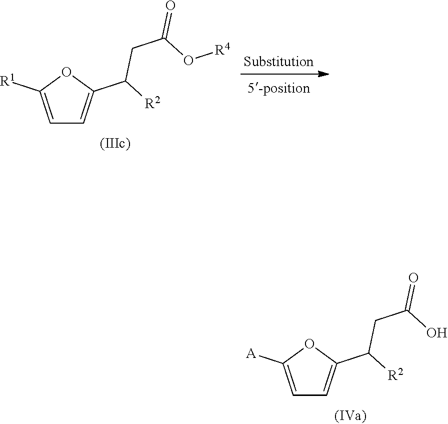 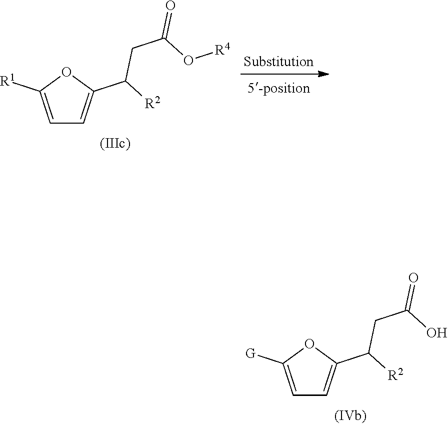 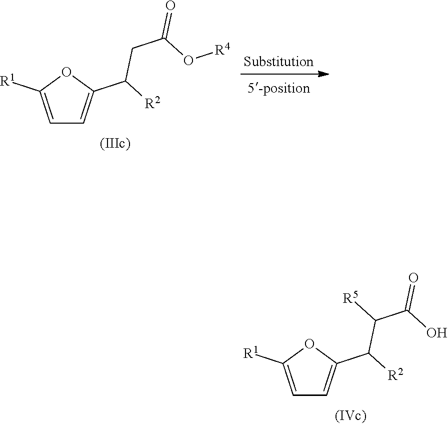
wherein for formula (IVa): A is an aliphatic chain comprising at least six carbon atoms and R² is H or an aliphatic group,
wherein for formula (IVb): G is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium, methylene sulfate, methylene sulfonate, methylene sulfinate, methylene thiosulfate, methylene sulfamidate, methylene carboxylate, methylene sarcosinate and taurate, methylene phosphate, methylene pyrophosphate, methylene phosphonate, methylene amines or ammonium, methylene polyammonium, methylene hydroxyammonium, methylene pyridinium, methylene picolinium, methylene imidazolinium, methylene benzimidazolinium, methylene oxonium, methylene sulfonium, and methylene phosphonium, poly(ethylene oxide), poly(co-ethylene oxide co-propylene oxide) (also referred to as poloxamers), polyglycosides, isosorbide and its derivatives, 1,4-sorbitane and its derivatives, and R² and R⁴ are independently H or an aliphatic group provided that at least one of R² and R⁴ is an aliphatic chain comprising more than 6 carbon atoms, and
wherein for formula (IVc): R⁵ is an aliphatic group and R¹ is H or CH₂OX, wherein X is H or an aliphatic group, and R² is H or an aliphatic group.

23. The method according to claim 18, said method comprising continuing the third-level compound, or a salt thereof, of formula (IIIc) in one or more steps to convert said third-level compound, or a salt thereof, to the surfactant, wherein said one or more steps comprise reacting said third-level compound, or a salt thereof, in a reaction comprising a decarboxylation to obtain the surfactant of formula and (IVb) 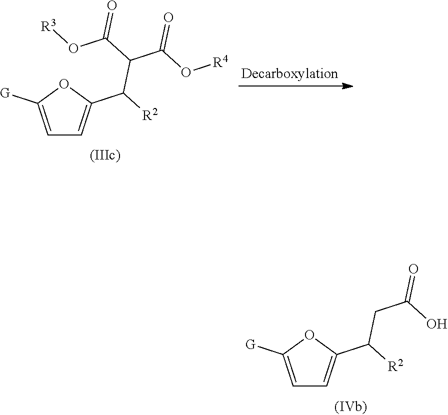
wherein G is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium, methylene sulfate, methylene sulfonate, methylene sulfinate, methylene thiosulfate, methylene sulfamidate, methylene carboxylate, methylene sarcosinate and taurate, methylene phosphate, methylene pyrophosphate, methylene phosphonate, methylene amines or ammonium, methylene polyammonium, methylene hydroxyammonium, methylene pyridinium, methylene picolinium, methylene imidazolinium, methylene benzimidazolinium, methylene oxonium, methylene sulfonium, and methylene phosphonium, poly(ethylene oxide), poly(co-ethylene oxide co-propylene oxide) (also referred to as poloxamers), polyglycosides, isosorbide and its derivatives, 1,4-sorbitane and its derivatives, and
wherein for formula (IVb): R² and R⁴ are independently H or an aliphatic group provided that at least one of R² and R⁴ is an aliphatic chain comprising more than 6 carbon atoms.

24. The method according to claim 21, said method comprising continuing the third-level compound, or a salt thereof, of formula (IIId) in one or more steps to convert said third-level compound, or a salt thereof, to the surfactant, wherein said one or more steps comprise reacting said third-level compound, or a salt thereof, in a reaction comprising a hydrogenation to obtain the surfactant of formula (IVa) 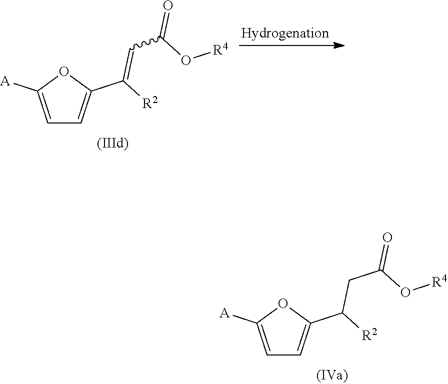
wherein A is an aliphatic chain comprising at least six carbon atoms,
wherein for formula (IVa) R² is H or an aliphatic group.

25. The method according to claim 20, said method comprising continuing the third-level compound, or a salt thereof, of formulae (IIIf) in one or more steps to convert said third-level compound, or a salt thereof, to the surfactant, wherein said one or more steps comprise reacting said third-level compound, or a salt thereof, in a reaction comprising a hydrogenation to obtain the surfactant of formula (IVb) 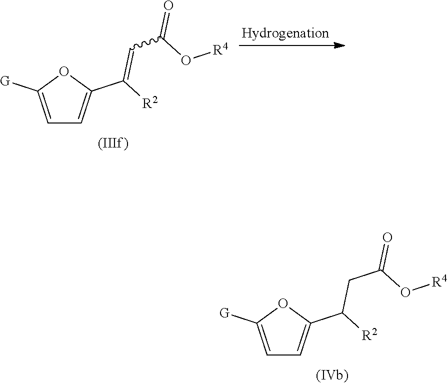
wherein G is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium, methylene sulfate, methylene sulfonate, methylene sulfinate, methylene thiosulfate, methylene sulfamidate, methylene carboxylate, methylene sarcosinate and taurate, methylene phosphate, methylene pyrophosphate, methylene phosphonate, methylene amines or ammonium, methylene polyammonium, methylene hydroxyammonium, methylene pyridinium, methylene picolinium, methylene imidazolinium, methylene benzimidazolinium, methylene oxonium, methylene sulfonium, and methylene phosphonium, poly(ethylene oxide), poly(co-ethylene oxide co-propylene oxide) (also referred to as poloxamers), polyglycosides, isosorbide and its derivatives, 1,4-sorbitane and its derivatives, and R² and R⁴ are independently H or an aliphatic group provided that at least one of R² and R⁴ is an aliphatic chain comprising more than 6 carbon atoms, and
wherein for formula (IVb): R² and R⁴ are independently H or an aliphatic group provided that at least one of R² and R⁴ is an aliphatic chain comprising more than 6 carbon atoms.

26. The method according to claim 17, said method comprising continuing the third-level compound, or a salt thereof, of formulae (IIIf) in one or more steps to convert said third-level compound, or a salt thereof, to the surfactant, wherein said one or more steps comprise reacting said third-level compound, or a salt thereof, in a reaction comprising a hydrogenation to obtain the surfactant of formula (IVb) 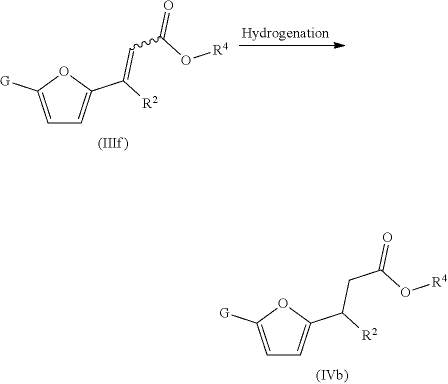
wherein G is selected from the group consisting of sulfate, sulfonate, sulfinate, thiosulfate, sulfamidate, carboxylate, sarcosinate and taurate, phosphate, pyrophosphate, phosphonate, amines or ammonium, polyammonium, hydroxyammonium, pyridinium, picolinium, imidazolinium, benzimidazolinium, oxonium, sulfonium, phosphonium, methylene sulfate, methylene sulfonate, methylene sulfinate, methylene thiosulfate, methylene sulfamidate, methylene carboxylate, methylene sarcosinate and taurate, methylene phosphate, methylene pyrophosphate, methylene phosphonate, methylene amines or ammonium, methylene polyammonium, methylene hydroxyammonium, methylene pyridinium, methylene picolinium, methylene imidazolinium, methylene benzimidazolinium, methylene oxonium, methylene sulfonium, and methylene phosphonium, poly(ethylene oxide), poly(co-ethylene oxide co-propylene oxide) (also referred to as poloxamers), polyglycosides, isosorbide and its derivatives, 1,4-sorbitane and its derivatives, and R² and R⁴ are independently H or an aliphatic group provided that at least one of R² and R⁴ is an aliphatic chain comprising more than 6 carbon atoms, and
wherein for formula (IVb): R² and R⁴ are independently H or an aliphatic group provided that at least one of R² and R⁴ is an aliphatic chain comprising more than 6 carbon atoms.

27. The method according to claim 19, said method comprising continuing the third-level compound, or a salt thereof, of formula (IIIb) in one or more steps to convert said third-level compound, or a salt thereof, to the surfactant, wherein said one or more steps comprise reacting said third-level compound, or a salt thereof, in a reaction comprising a decarboxylation to obtain the surfactant of formula (IVa) 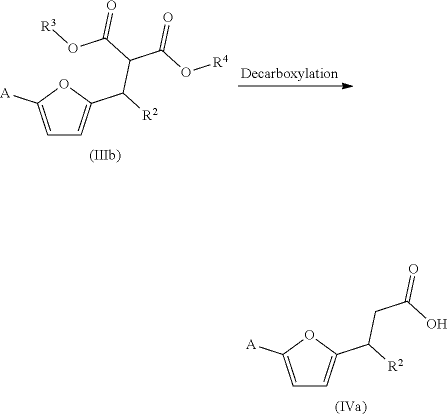
wherein A is an aliphatic chain comprising at least six carbon atoms, and
wherein for formula (IVa) R² is H or an aliphatic group.
